(19) United States
(12) Patent Application Publication
(10) Pub. No.: US 2023/0399657 A1
(43) Pub. Date: Dec. 14, 2023

(54) LIVER DE-TARGETED RECOMBINANT AAV CAPSID PROTEINS

(71) Applicant: Homology Medicines, Inc., Bedford, MA (US)

(72) Inventors: Laura Jane SMITH, Bedford, MA (US); Laura Van Lieshout, Bedford, MA (US); Lindsay Schulman, Bedford, MA (US); Omar Francone, Bedford, MA (US)

(21) Appl. No.: 18/319,052

(22) Filed: May 17, 2023

Related U.S. Application Data
(60) Provisional application No. 63/449,772, filed on Mar. 3, 2023.
(60) Provisional application No. 63/365,989, filed on Jun. 7, 2022.
(60) Provisional application No. 63/364,839, filed on May 17, 2022.

(51) Int. Cl. C12N 15/86; C07K 14/005

(52) U.S. Cl. CPC C12N 15/86; C07K 14/005; C12N 2750/14152; C12N 2750/14171; C12N 2750/14122; C12N 2750/14145; C12N 2750/14143

(57) ABSTRACT

Provided herein are recombinant adeno-associated virus (AAV) capsid proteins, compositions (e.g., rAAV) comprising the capsid proteins, nucleic acids encoding the capsid proteins, and methods of making and using the capsid proteins.

### RELATED APPLICATIONS

This application claims priority to U.S. Provisional Patent Application Ser. Nos. 63/364,839, filed May 17, 2022; 63/365,989, filed Jun. 7, 2022; and 63/449,772, filed Mar. 3, 2023, the entire disclosures of which are hereby incorporated herein by reference.

### SEQUENCE LISTING

This application contains a sequence listing which has been submitted electronically in ST.26 format and is hereby incorporated by reference in its entirety (said ST.26 copy, created on May 16, 2023, is named “199936_seglist.xml” and is 2,064,607 bytes in size).

### BACKGROUND

Adeno-associated viruses (AAVs) are small, non-enveloped viruses of the genus Dependoviruses within the family Parvoviridae. They package a single-stranded DNA genome that is approximately 4.7 kb in length, consisting of rep and cap genes flanked by inverted terminal repeat regions (ITRs). AAVs are an ideal delivery system for gene therapy because AAV infection of cells in culture is non-cytopathic, and natural infection of humans and other animals is silent. Further, because the signals directing AAV replication, genome encapsidation and integration are contained within the inverted terminal repeats (ITRs) of the AAV genome, essentially all of the internal 4.7 kb of the AAV genome (encoding the replication and structural capsid proteins, rep-cap) can be replaced with heterologous nucleic acid sequences, such as a transgene for effecting gene therapy.

Recombinant AAV (rAAV) gene therapy vectors typically consist of a recombinant AAV genome comprising a gene therapy transgene, packaged within an AAV capsid. The AAV capsid, which is formed by an assembly of 60 AAV capsid proteins, interacts with the cell surface of target cells and determines the tropism and transduction efficiency of rAAV. For tissue-specific genetic diseases, it desirable to have rAAVs that could specifically and efficiently target tissues in need of gene therapy, while at the same time de-target certain tissues.

Accordingly, there is a need in the art for tissue-specific AAV capsids for use in gene therapy applications.

### SUMMARY

Provided herein are recombinant adeno-associated virus (AAV) capsid proteins, compositions (e.g., rAAV) comprising the capsid proteins, nucleic acids encoding the capsid proteins, and methods of making and using the capsid proteins. The recombinant AAV capsid proteins provided herein are engineered to comprise non-wild type amino acids at positions corresponding to amino acid positions 346, 501, 505, and/or 706 of SEQ ID NO: 14. Also provided are recombinant AAV capsid proteins wherein the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. These recombinant AAV capsid proteins demonstrate reduced transduction of liver cells compared to the parental AAV capsid proteins. These recombinant AAV capsid proteins also result in increased expression of payload in transduced cells.

Accordingly, in one aspect, the instant disclosure provides a recombinant adeno-associated virus (AAV) capsid protein, wherein the amino acid in the capsid protein corresponding to amino acid positions 346, 501, 505, and/or 706 of SEQ ID NO: 14 is not the wild type amino acid.

In one aspect, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the amino acid in the capsid protein corresponding to amino acid position 501 of SEQ ID NO: 14 is isoleucine, and the amino acid in the capsid protein corresponding to amino acid position 505 of SEQ ID NO: 14 is arginine. In certain embodiments, the amino acid in the capsid protein corresponding to amino acid position 501 of SEQ ID NO: 14 is isoleucine, and the amino acid in the capsid protein corresponding to amino acid position 706 of SEQ ID NO: 14 is cysteine. In certain embodiments, the amino acid in the capsid protein corresponding to amino acid position 505 of SEQ ID NO: 14 is arginine, and the amino acid in the capsid protein corresponding to amino acid position 706 of SEQ ID NO: 14 is cysteine. In certain embodiments, the amino acid in the capsid protein corresponding to amino acid position 501 of SEQ ID NO: 14 is isoleucine, the amino acid in the capsid protein corresponding to amino acid position 505 of SEQ ID NO: 14 is arginine, and the amino acid in the capsid protein corresponding to amino acid position 706 of SEQ ID NO: 14 is cysteine. In certain embodiments, the amino acid in the capsid protein corresponding to amino acid position 346 of SEQ ID NO: 14 is alanine, the amino acid in the capsid protein corresponding to amino acid position 501 of SEQ ID NO: 14 is isoleucine, and the amino acid in the capsid protein corresponding to amino acid position 505 of SEQ ID NO: 14 is arginine. In certain embodiments, the amino acid in the capsid protein corresponding to amino acid position 346 of SEQ ID NO: 14 is alanine, the amino acid in the capsid protein corresponding to amino acid position 501 of SEQ ID NO: 14 is isoleucine, and the amino acid in the capsid protein corresponding to amino acid position 706 of SEQ ID NO: 14 is cysteine. In certain embodiments, the amino acid in the capsid protein corresponding to amino acid position 346 of SEQ ID NO: 14 is alanine, the amino acid in the capsid protein corresponding to amino acid position 505 of SEQ ID NO: 14 is arginine, and the amino acid in the capsid protein corresponding to amino acid position 706 of SEQ ID NO: 14 is cysteine. In certain embodiments, the amino acid in the capsid protein corresponding to amino acid position 346 of SEQ ID NO: 14 is alanine, the amino acid in the capsid protein corresponding to amino acid position 501 of SEQ ID NO: 14 is isoleucine, the amino acid in the capsid protein corresponding to amino acid position 505 of SEQ ID NO: 14 is arginine, and the amino acid in the capsid protein corresponding to amino acid position 706 of SEQ ID NO: 14 is cysteine.

In certain embodiments, the recombinant AAV capsid protein is a variant of a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein.

In certain embodiments, the recombinant AAV capsid protein comprises an amino acid sequence that is 95% identical to any one of the amino acid sequences of SEQ ID NOs: 1-30. In certain embodiments, the recombinant AAV capsid protein comprises any one of the amino acid sequences of SEQ ID NOs: 31-411 and 453-1304.

In one aspect, the instant disclosure provides an isolated polynucleotide encoding a recombinant AAV capsid protein as described herein. In certain embodiments, a vector comprises the polynucleotide. In certain embodiments, the vector is a plasmid or a viral vector. In certain embodiments, the viral vector is a retrovirus vector, a herpes virus vector, a baculovirus vector, or an adenovirus vector. In certain embodiments, the vector is an expression vector.

In one aspect, the instant disclosure provides a recombinant cell comprising a polynucleotides or vector as described herein.

In one aspect, the instant disclosure provides a method of producing a recombinant AAV capsid protein, the method comprising culturing the recombinant cell under conditions where the polynucleotide is expressed, and the capsid protein is produced.

In one aspect, the instant disclosure provides a recombinant adeno-associated virus (rAAV) comprising: (a) a capsid comprising one or more of the recombinant AAV capsid protein of any one of claims 1-13; and (b) an rAAV genome.

In certain embodiments, the rAAV genome comprises a transgene. In certain embodiments, the transgene encodes a polypeptide. In certain embodiments, the transgene encodes a miRNA, shRNA, siRNA, antisense RNA, gRNA, antagomir, miRNA sponge, RNA aptazyme, RNA aptamer, lncRNA, ribozyme, or mRNA. In certain embodiments, the transgene is operably linked to a transcriptional regulatory element.

In certain embodiments, the rAAV genome comprises an editing genome.

In one aspect, the instant disclosure provides a method for transducing a cell, the method comprising contacting the cell with an rAAV described herein under conditions whereby the cell is transduced.

In one aspect, the instant disclosure provides a method for delivering a transgene in a cell, the method comprising contacting the cell with an rAAV described herein under conditions whereby the cell is transduced and the transgene is expressed.

In one aspect, the instant disclosure provides a method for editing a target locus in a genome of a cell, the method comprising contacting an rAAV described herein (e.g., comprising an rAAV genome comprising an editing genome) under conditions whereby the cell is transduced and the target locus is edited.

In certain embodiments, the cell is in a subject and the rAAV is administered to the subject. In certain embodiments, the rAAV is administered to the subject intravenously, intraperitoneally, subcutaneously, intramuscularly, intrathecally, or intradermally. In certain embodiments, the subject is a human subject.

In one aspect, the instant disclosure provides a packaging system for preparation of an rAAV, wherein the packaging system comprises: (a) a first nucleotide sequence encoding one or more AAV Rep proteins; (b) a second nucleotide sequence encoding the recombinant AAV capsid protein of any one of claims 1-13; and (c) a third nucleotide sequence comprising an rAAV genome sequence.

In certain embodiments, the packaging system comprises a first vector comprising the first nucleotide sequence and the second nucleotide sequence, and a second vector comprising the third nucleotide sequence. In certain embodiments, the packaging system further comprises a fourth nucleotide sequence comprising one or more helper virus genes, optionally wherein the fourth nucleotide sequence is comprised within a third vector. In certain embodiments, the fourth nucleotide sequence comprises one or more genes from a virus selected from the group consisting of adenovirus, herpesvirus, vaccinia virus, and cytomegalovirus (CMV). In certain embodiments, the first vector, second vector, and/or the third vector is a plasmid.

In certain embodiments, the packaging system comprises a first vector comprising the first nucleotide sequence, the second nucleotide sequence, and the third nucleotide sequence. In certain embodiments, the packaging system further comprises a fourth nucleotide sequence comprising one or more helper virus genes, optionally wherein the fourth nucleotide sequence is comprised within a second vector. In certain embodiments, the fourth nucleotide sequence comprises one or more genes from a virus selected from the group consisting of adenovirus, herpesvirus, vaccinia virus, and cytomegalovirus (CMV). In certain embodiments, the first vector and/or second vector is a plasmid.

In one aspect, the instant disclosure provides a method for recombinant preparation of an rAAV, the method comprising introducing a packaging system described herein into a cell under conditions whereby the rAAV is produced.

In one aspect, the instant disclosure provides an rAAV described herein for use in medicine, for use as therapy, or for use as a medicament.

### BRIEF DESCRIPTION OF THE DRAWINGS

FIG. 1 is an alignment of amino acid sequences of capsid proteins of various serotypes. From top to bottom: AAV1 (SEQ ID NO: 16), AAV2 (SEQ ID NO: 17), AAV8 (SEQ ID NO: 21), AAV5 (SEQ ID NO: 19), AAV6 (SEQ ID NO: 20), AAV3B (SEQ ID NO: 18), AAVhu.32 (SEQ ID NO: 23), AAVhu.37 (SEQ ID NO: 24), AAV-DJ (SEQ ID NO: 26), AAVrh.74 (SEQ ID NO: 25), AAV Anc80L65 (SEQ ID NO: 27), AAV Oligo100 (SEQ ID NO: 28), AAV9 (SEQ ID NO: 22), AAV LK03 (SEQ ID NO: 30), AAVrh.10 (SEQ ID NO: 29), AAVHSC16 (SEQ ID NO: 14).

FIGS. 2A-2D is a schematic showing cell surface glycosidic chains associated with CHO cell mutants (FIG. 2A) and graphs showing the binding and transduction of AAVHSCs in Lec2 cells with terminally exposed galactose (FIGS. 2B-2D). FIG. 2B is a graph showing vector genomes bound to cell surface of Pro5 and Lec2 cells at time of transduction. FIG. 2C is a graph showing GFP expression at 24 hours. FIG. 2D is a graph showing the fold change in cell surface binding between Lec2 and Pro5 cells. Statistical analysis was performed using a two-way analysis of variance (ANOVA). ns indicates no statistical significance and **** indicates statistical significance at p<0.0001. All transductions were performed with biological triplicates and all molecular analysis was performed with technical triplicates.

FIGS. 3A-3B are graphs showing terminal galactose binding on AAVHSC16, AAVHSC15, and AAVHSC15 variants. FIG. 3A is a graph showing cellular surface bound vector genomes. FIG. 3B is a graph showing GFP expression at 24 hours. All vectors were tested on all CHO cell lines, and lack of visible bars indicate little to no surface bound VGs or GFP expression from transduction. Statistical analysis was performed using a two-way analysis of variance (ANOVA). ns indicates no statistical significance, * indicates statistical significance at p<0.05, ** indicates statistical significance at p<0.01, and **** indicates statistical significance at p<0.0001. All transductions were performed with biological triplicates and all molecular analysis was performed with technical triplicates.

FIGS. 4A-4B are graphs showing the effect of all naturally occurring unique residues on AAVHSC15 and AAVHSC16 on the binding and expression profile in CHO cells. FIG. 4A is a graph showing cellular surface bound vector genomes. FIG. 4B is a graph showing GFP expression at 24 hours. All vectors were tested on all CHO cell lines and lack of visible bars indicate little to no surface bound VGs or GFP expression from transduction. Statistical analysis was performed using a two-way analysis of variance (ANOVA). ns indicates no statistical significance, and **** indicates statistical significance at p<0.0001. All transductions were performed with biological triplicates and all molecular analysis was performed with technical triplicates.

FIGS. 5A-5B are graphs showing that the effect of the amino acid at residue 501 on overall binding of AAVHSC16. FIG. 5A is a graph showing cellular surface bound vector genomes. FIG. 5B is a graph showing GFP expression at 24 hours. All vectors were tested on all CHO cell lines and lack of visible bars indicate little to no surface bound VGs or GFP expression from transduction.

FIGS. 6A-6E are graphs showing the biodistribution of AAV2, AAV9, AAVHSC15, AAVHSC16, AAVHSC15 F501I, and/or AAVHSC15 Y706C. FIG. 6A is a graph showing total body transgene expression as determined by whole body bioluminescence imaging. FIGS. 6B and 6C are graphs showing tissue-specific transgene expression determined by tissue-specific bioluminescence at time of harvest for mice injected at the 1×10¹³ or 1×10¹⁴ vg/kg dose, respectively. In FIG. 6B, the bars indicate, from left to right for each tissue, tissue-specific transgene expression in mice injected with AAV2, AAV9, AAVHSC15, AAVHSC16, AAVHSC15 F501I, and AAVHSC15 Y706C. In FIG. 6C, the bars indicate, from left to right for each tissue, tissue-specific transgene expression in mice injected with AAV2, AAV9, AAVHSC15, AAVHSC16, and AAVHSC15 F501I. FIGS. 6D and 6E are graphs showing tissue-specific vgs as determined by ddPCR for mice injected at the 1×10¹³ or 1×10¹⁴ vg/kg dose, respectively. In FIG. 6D, the bars indicate, from left to right for each tissue, tissue-specific transgene expression in mice injected with AAV2, AAV9, AAVHSC15, AAVHSC16, AAVHSC15 F501I, and AAVHSC15 Y706C. In FIG. 6E, the bars indicate, from left to right for each tissue, tissue-specific transgene expression in mice injected with AAV2, AAV9, AAVHSC15, AAVHSC16, and AAVHSC15 F501I. Statistical analysis was determined by two-way ANOVA. ns indicates no statistical significance, * indicates statistical significance at p<0.05, ** indicates statistical significance at p<0.01, and *** indicates statistical significance at p<0.001. N=3-5 animals per group.

FIG. 7 is a graph showing the transduction of AAV2, AAV9, AAVHSC15, AAVHSC16, AAVHSC15 F501I, and/or AAVHSC15 Y706C in various tissues. FIG. 7 is a graph showing vector genomes in specified tissues. Only significant differences in transduction by vgs were observed in the heart at this dose, statistical analysis performed by two-way ANOVA. N=3 animals per group. In FIG. 7, the bars indicate, from left to right for each tissue, vector genomes per allele in tissues of mice injected with AAV2, AAV9, AAVHSC15, AAVHSC16, AAVHSC15 F501I, and AAVHSC15 Y706C.

FIGS. 8A-8G are graphs showing the biodistribution of AAVHSC16 in NHP in vivo and the effect on ALT and AST levels after high dose IV injection of AAVHSC16. FIG. 8A is a graph showing vector genome measurements from tissue lysates. FIG. 8B is a graph showing ALT levels in NHPs dosed at 7×10¹³ vg/kg with AAVHSC16 scCBA.eGFP vector or vehicle assessed prior to dosing followed by 1 and 2 weeks post dosing. FIG. 8C is a graph showing AST levels in NHPs dosed at 7×10¹³ vg/kg with AAVHSC16 scCBA.eGFP vector or vehicle assessed prior to dosing followed by 1 and 2 weeks post dosing. FIG. 8D is a graph showing ALT and AST levels in NHPs dosed at 1×10¹⁴ vg/kg with AAVHSC16 ssDNG.PAH vector assessed prior to dosing followed by 3, 14, and 28 days post dosing. FIG. 8E is a graph showing ALT levels in NHPs dosed at 4.2×10¹³ to 7×10¹³ vg/kg with AAVHSC8, AAVHSC16, and AAVHSC17 scCBA.eGFP vector or vehicle were assessed at 2 weeks post dosing. FIG. 8F is a graph showing AST levels in NHPs dosed at 4.2×10¹³ to 7×10¹³ vg/kg with AAVHSC8, AAVHSC16, and AAVHSC17 scCBA.eGFP vector or vehicle were assessed at 2 weeks post dosing. FIG. 8G is a graph showing ALT and AST levels in NHPs dosed at 1×10¹⁴ vg/kg with AAV9, AAVHSC8, AAVHSC16, and AAVHSC17 ssDNG.PAH vector assessed at 3 days post dosing. Statistical analysis was determined by one-way ANOVA. ns indicates no statistical significance, and ****=p<0.0001. N=2 animals per group.

FIGS. 9A-9B are graphs showing the whole blood and serum clearance of mice administered AAVHSC15 and AAVHSC16. FIG. 9A is a graph showing vector genomes quantified in whole blood by qPCR of mice administered AAVHSC15, AAVHSC16, AAVHSC15-F501I, and AAVHSC16-Y706C. FIG. 9B is a graph showing vector genomes quantified in whole blood and serum by qPCR of mice administered AAVHSC15, AAVHSC16 scCBA-GFP vectors. B6 albino mice were serial bled (2 min, 30 min, 1 hr, 3 hr, 8 hr, 24 hr, and 48 hr).

FIG. 10 is a graph showing the transduction of indicated vectors in heart, liver, gastrocnemius, brain, and spinal cord cells. In FIG. 10, the bars indicate, from left to right for each tissue, vector genomes per allele in tissues of mice injected with AAVHSC15, AAVHSC7, AAVHSC7 G505R, AAVHSC8, and AAVHSC8 G505R.

FIG. 11 is a graph showing the transduction of indicated vectors in heart, liver, gastrocnemius, brain, and spinal cord cells. In FIG. 11, the bars indicate, from left to right for each tissue, vector genomes per allele in tissues of mice injected with AAVHSC15, AAVHSC16 T346A, AAVHSC16 I501F, AAVHSC16, AAVHSC16 I501YB, and AAVHSC16 I501W.

FIGS. 12A-12C are graphs showing the transduction of indicated vectors in heart and liver cells. Transduction efficiency was measured as a function of vector genomes quantified in transduced rat H9C2 cells (FIG. 12A), human Ac16 cells (FIG. 12B), and primary human hepatocytes (FIG. 12C). In FIG. 12C, the bars indicate, from left to right for each tissue, vector genomes per allele in primary human hepatocytes transduced with the indicated vectors at multiplicities of infection (MOI) of 1.5e5, 3e5, and 6e5.

FIGS. 13A-13E are graphs showing the transduction of indicated vectors in heart, muscle, and liver cells. Transduction efficiency was measured as a function of vector genomes quantified in heart cells (FIG. 13A), gastrocnemius cells (FIG. 13B), and liver cells (FIG. 13C).

FIG. 13D and FIG. 13E are graphs showing the ratio of transduction efficiency of the indicated vectors of heart cells or gastrocnemius cells, respectively, to the transduction efficiency of the indicated vectors of liver cells.

FIGS. 14A and 14B are graphs showing the ability of AAVHSC16 to transduce various tissues in mice intravenously administered AAVHSC16 vector at the indicated doses. FIG. 14A shows the transduction efficiency as a ratio between the level of transduction of various tissues as indicated, to the level of transduction of liver tissue. Ratios were calculated by dividing the average vgs/allele detected in a specific tissue of 4-5 mice by the average vgs/allele detected in the liver of the same mice. In FIG. 14A, the dots indicate, from left to right for each dose, the ratios between the level of transduction of the heart, lung, brain, kidney, spleen, and trigeminal ganglion, to the level of transduction of liver tissue. FIG. 14B shows the transduction efficiency of AAVHSC16 in the heart and liver of 4-5 mice, presented as the average transcripts per vg per cell of each mouse per group. In FIG. 14B, for each dose, the left and right dots indicate the transduction efficiency of the heart and liver, respectively.

FIGS. 15A and 15B are graphs showing the level of I2S transcripts detected in the heart and liver, respectively, of mice intravenously administered a rAAV comprising a transgene encoding IDS packaged in AAVHSC16. Bars indicate the average transcripts per ng of RNA of 4-5 mice.

FIGS. 16A and 16B are graphs showing the ability of AAVHSC16 to express protein in the liver and secrete protein into the circulation of mice intravenously administered a rAAV comprising a transgene encoding IDS packaged in AAVHSC16, at the indicated doses. FIG. 16A shows the level of I2S activity in the liver as fold over wild-type levels, determined by dividing the I2S activity detected in the liver of mice administered the AAVHSC16 vector by the average I2S activity detected in the liver of control mice (administered formulation buffer control). FIG. 16B shows the level of I2S activity in the serum as fold over wild-type levels, determined by dividing the I2S activity detected in the serum of mice administered the AAVHSC16 vector by the average I2S activity detected in the serum of control mice. 4-5 mice were used in each condition.

### DETAILED DESCRIPTION

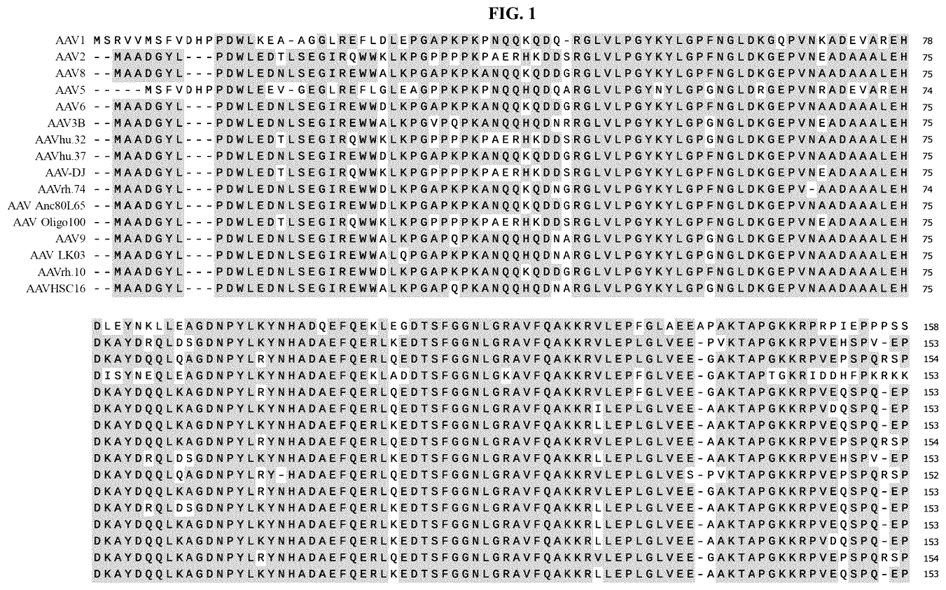

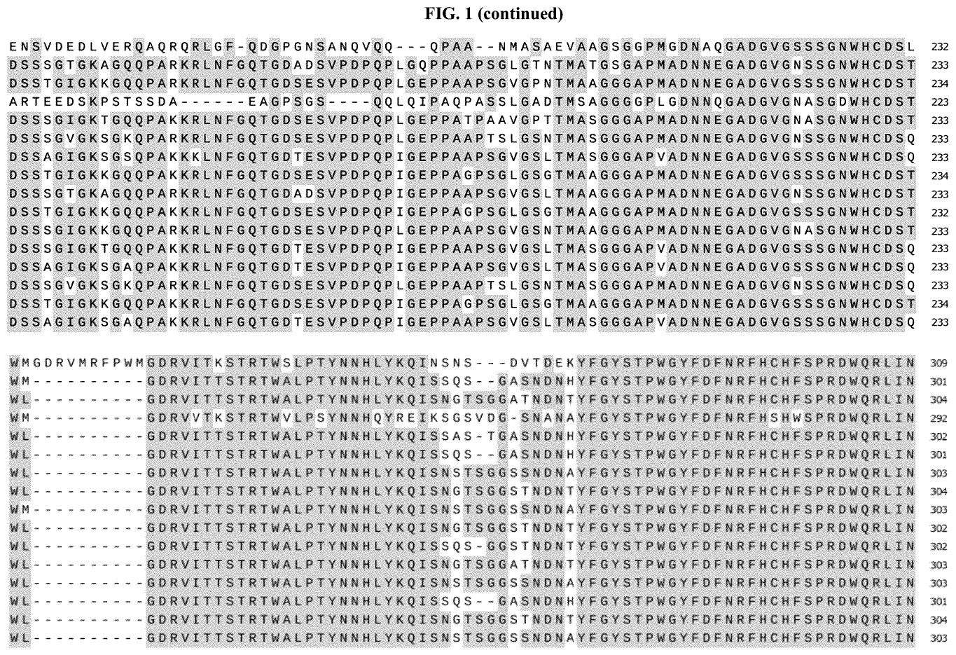

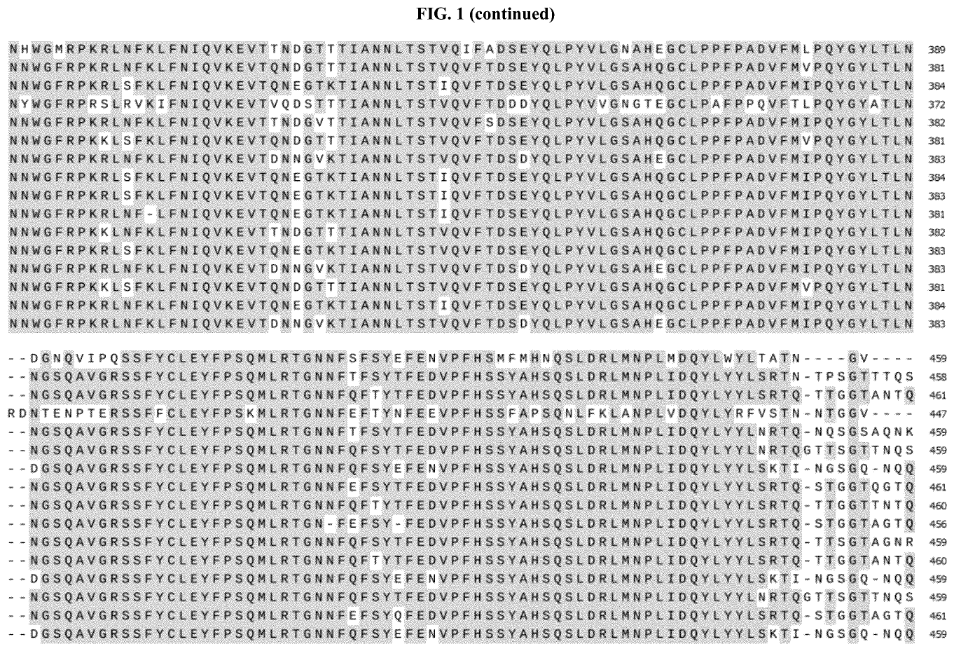

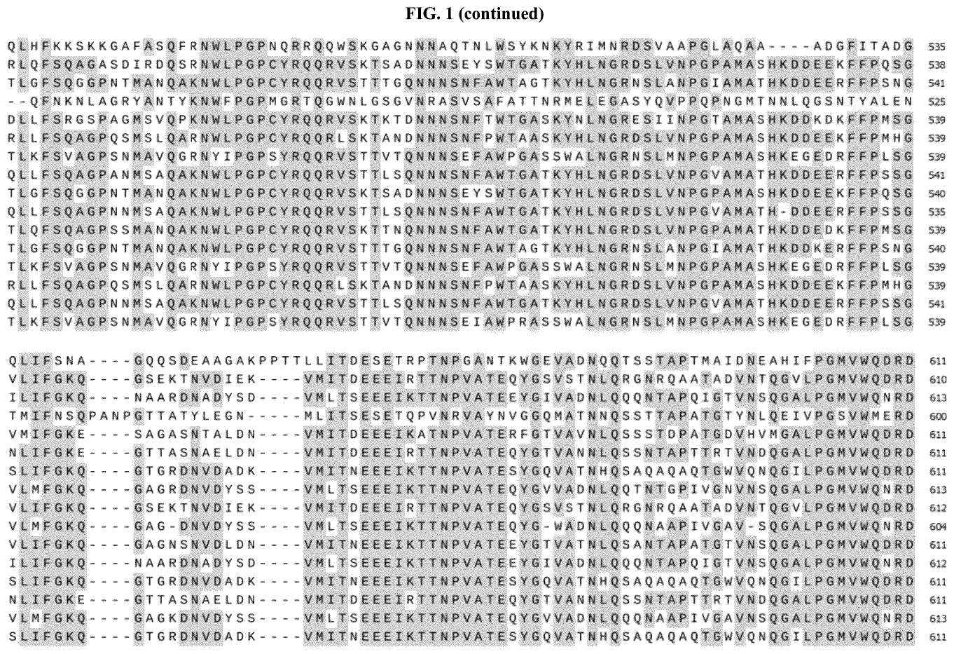

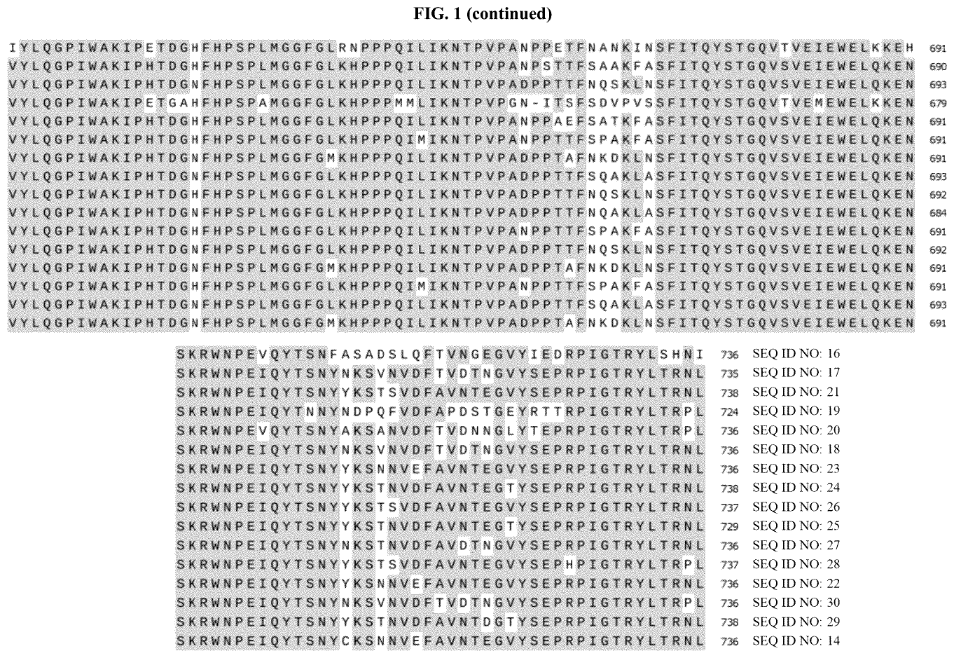

Provided herein are recombinant adeno-associated virus (AAV) capsid proteins, compositions (e.g., rAAV) comprising the capsid proteins, nucleic acids encoding the capsid proteins, and methods of making and using the capsid proteins. The recombinant AAV capsid proteins provided herein are engineered to comprise non-wild type amino acids at positions corresponding to amino acid positions 346, 501, 505, and/or 706 of SEQ ID NO: 14. Also provided are recombinant AAV capsid proteins wherein the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. These recombinant AAV capsid proteins demonstrate reduced transduction of liver cells compared to the parental AAV capsid proteins. These recombinant AAV capsid proteins also result in increased expression of payload in transduced cells. As shown in FIG. 1, the amino acids at positions 346, 501, 505, and 706 of AAV capsid proteins of various serotypes are well conserved.

### I. DEFINITIONS

As used herein, the term “AAV” is a standard abbreviation for adeno-associated virus.

As used herein, the term “recombinant adeno-associated virus” or “rAAV” refers to an AAV comprising a genome lacking functional rep and cap genes.

As used herein, the term “cap gene” refers to a nucleic acid sequence that encodes a capsid protein. For AAV, the capsid protein may be VP1, VP2, or VP3. VP1, VP2, and/or VP3 capsid proteins assemble into a capsid that surrounds the rAAV genome.

As used herein, the term “rep gene” refers to the nucleic acid sequences that encode the non-structural proteins (e.g., rep78, rep68, rep52, and rep40) required for the replication and production of an AAV.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., a polypeptide encoding a therapeutic protein operably linked to a transcriptional regulatory element (i.e., payload)), the rAAV genome can be in the sense or antisense orientation relative to the direction of transcription of the transgene.

As used herein, an “isolated polynucleotide” refers to a polynucleotide that has been separated from one or more nucleic acid molecules present in the natural source of the polynucleotide.

As used herein, the term “editing genome” refers to an rAAV genome, comprising an editing element for editing a target locus, flanked by (i) a 5′ homology arm sequence 5′ of the editing element having homology to a first genomic region 5′ to the target locus; and (ii) a 3′ homology arm nucleotide 3′ of the editing element having homology to a second genomic region 3′ to the target locus. An editing genome is capable of integrating an editing element via homologous recombination into a target locus (e.g., a human target locus) to edit that locus (e.g., to correct a genetic defect in a gene). The skilled artisan will appreciate that the portion of an editing genome comprising a 5′ homology arm, editing element, a 3′ homology arm can be in the sense or antisense orientation relative to the target locus.

As used herein, the term “editing element” refers to the portion of an editing genome that when integrated by homologous recombination at a target locus modifies the target locus. An editing element can mediate insertion, deletion, or substitution of one or more nucleotides at the target locus.

As used herein, the term “target locus” refers to a region of a chromosome or an internucleotide bond (e.g., a region or an internucleotide bond of a human gene) that is modified by an editing element.

As used herein, the term “homology arm” refers to a portion of an editing genome positioned 5′ or 3′ of an editing element that is substantially identical (e.g., 100% identical) to a portion of the genome sequence flanking a target locus. In certain embodiments, the target locus is in a human gene, and the homology arm comprises a sequence substantially identical to the genome sequence flanking the target locus in the human gene. In certain embodiments, the target locus in an intergenic region of a genome (e.g., human genome).

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, a “vector” refers to a nucleic acid molecule that is a vehicle for introducing a nucleic acid molecule (e.g., a polynucleotide disclosed herein) into a cell.

As used herein, an “expression vector” refers to a vector comprising transcriptional regulatory elements operably linked to a gene of interest (e.g., a polynucleotide disclosed herein) that facilitate the expression of the gene of interest in a cell and/or a cell free expression system.

As used herein, the term “transgene” refers to a non-AAV nucleic acid sequence that encodes a polypeptide (e.g., an antibody or scFv) or non-coding RNA (e.g., a miRNA, shRNA, siRNA, antisense RNA, gRNA, antagomir, miRNA sponge, RNA aptazyme, or RNA aptamer).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a polynucleotide sequence (e.g., a transgene disclosed herein) to be transcribed. Typically, gene expression is placed under the control of a TRE comprising one or more promoter and/or enhancer elements. The transgene is “operably linked” to the TRE if the transcription of the transgene is controlled or influenced by the TRE. The promoter and enhancer elements of the TRE may be in any orientation and/or distance from the transgene, as long as the desired transcriptional activity is obtained. In an embodiment, the TRE is upstream from the transgene.

As used herein, the term “effective amount” in the context of the administration of an AAV to a subject refers to the amount of the AAV that achieves a desired prophylactic or therapeutic effect.

### II. AAV CAPSID PROTEINS

In one aspect, the instant disclosure provides a recombinant adeno-associated virus (AAV) capsid protein, wherein the amino acid in the capsid protein corresponding to amino acid positions 346, 501, 505, 705, and/or 706 of SEQ ID NO: 14 is not the wild type amino acid (i.e., not the amino acid that occurs in nature). In certain embodiments, the instant disclosure provides a recombinant AAV capsid protein, wherein the amino acid in the capsid protein corresponding to amino acid position 346 of SEQ ID NO: 14 is not the wild type amino acid. In certain embodiments, the instant disclosure provides a recombinant AAV capsid protein, wherein the amino acid in the capsid protein corresponding to amino acid position 501 of SEQ ID NO: 14 is not the wild type amino acid. In certain embodiments, the instant disclosure provides a recombinant AAV capsid protein, wherein the amino acid in the capsid protein corresponding to amino acid position 505 of SEQ ID NO: 14 is not the wild type amino acid. In certain embodiments, the instant disclosure provides a recombinant AAV capsid protein, wherein the amino acid in the capsid protein corresponding to amino acid position 705 of SEQ ID NO: 14 is not the wild type amino acid. In certain embodiments, the instant disclosure provides a recombinant AAV capsid protein, wherein the amino acid in the capsid protein corresponding to amino acid position 706 of SEQ ID NO: 14 is not the wild type amino acid.

In certain embodiments, the instant disclosure provides a recombinant AAV capsid protein, wherein the amino acid in the capsid protein corresponding to amino acid positions 346 and 501 of SEQ ID NO: 14 is not the wild type amino acid. In certain embodiments, the instant disclosure provides a recombinant AAV capsid protein, wherein the amino acid in the capsid protein corresponding to amino acid positions 346 and 505 of SEQ ID NO: 14 is not the wild type amino acid. In certain embodiments, the instant disclosure provides a recombinant AAV capsid protein, wherein the amino acid in the capsid protein corresponding to amino acid positions 346 and 705 of SEQ ID NO: 14 is not the wild type amino acid. In certain embodiments, the instant disclosure provides a recombinant AAV capsid protein, wherein the amino acid in the capsid protein corresponding to amino acid positions 346 and 706 of SEQ ID NO: 14 is not the wild type amino acid. In certain embodiments, the instant disclosure provides a recombinant AAV capsid protein, wherein the amino acid in the capsid protein corresponding to amino acid positions 501 and 505 of SEQ ID NO: 14 is not the wild type amino acid. In certain embodiments, the instant disclosure provides a recombinant AAV capsid protein, wherein the amino acid in the capsid protein corresponding to amino acid positions 501 and 705 of SEQ ID NO: 14 is not the wild type amino acid. In certain embodiments, the instant disclosure provides a recombinant AAV capsid protein, wherein the amino acid in the capsid protein corresponding to amino acid positions 501 and 706 of SEQ ID NO: 14 is not the wild type amino acid. In certain embodiments, the instant disclosure provides a recombinant AAV capsid protein, wherein the amino acid in the capsid protein corresponding to amino acid positions 505 and 705 of SEQ ID NO: 14 is not the wild type amino acid. In certain embodiments, the instant disclosure provides a recombinant AAV capsid protein, wherein the amino acid in the capsid protein corresponding to amino acid positions 505 and 706 of SEQ ID NO: 14 is not the wild type amino acid. In certain embodiments, the instant disclosure provides a recombinant AAV capsid protein, wherein the amino acid in the capsid protein corresponding to amino acid positions 705 and 706 of SEQ ID NO: 14 is not the wild type amino acid.

In certain embodiments, the instant disclosure provides a recombinant AAV capsid protein, wherein the amino acid in the capsid protein corresponding to amino acid positions 346, 501, and 505 of SEQ ID NO: 14 is not the wild type amino acid. In certain embodiments, the instant disclosure provides a recombinant AAV capsid protein, wherein the amino acid in the capsid protein corresponding to amino acid positions 346, 501, and 705 of SEQ ID NO: 14 is not the wild type amino acid. In certain embodiments, the instant disclosure provides a recombinant AAV capsid protein, wherein the amino acid in the capsid protein corresponding to amino acid positions 346, 501, and 706 of SEQ ID NO: 14 is not the wild type amino acid. In certain embodiments, the instant disclosure provides a recombinant AAV capsid protein, wherein the amino acid in the capsid protein corresponding to amino acid positions 346, 505, and 705 of SEQ ID NO: 14 is not the wild type amino acid. In certain embodiments, the instant disclosure provides a recombinant AAV capsid protein, wherein the amino acid in the capsid protein corresponding to amino acid positions 346, 505, and 706 of SEQ ID NO: 14 is not the wild type amino acid. In certain embodiments, the instant disclosure provides a recombinant AAV capsid protein, wherein the amino acid in the capsid protein corresponding to amino acid positions 346, 705, and 706 of SEQ ID NO: 14 is not the wild type amino acid. In certain embodiments, the instant disclosure provides a recombinant AAV capsid protein, wherein the amino acid in the capsid protein corresponding to amino acid positions 501, 505, and 705 of SEQ ID NO: 14 is not the wild type amino acid. In certain embodiments, the instant disclosure provides a recombinant AAV capsid protein, wherein the amino acid in the capsid protein corresponding to amino acid positions 501, 505, and 706 of SEQ ID NO: 14 is not the wild type amino acid. In certain embodiments, the instant disclosure provides a recombinant AAV capsid protein, wherein the amino acid in the capsid protein corresponding to amino acid positions 501, 705, and 706 of SEQ ID NO: 14 is not the wild type amino acid. In certain embodiments, the instant disclosure provides a recombinant AAV capsid protein, wherein the amino acid in the capsid protein corresponding to amino acid positions 505, 705, and 706 of SEQ ID NO: 14 is not the wild type amino acid.

In certain embodiments, the instant disclosure provides a recombinant AAV capsid protein, wherein the amino acid in the capsid protein corresponding to amino acid positions 346, 501, 505, and 705 of SEQ ID NO: 14 is not the wild type amino acid. In certain embodiments, the instant disclosure provides a recombinant AAV capsid protein, wherein the amino acid in the capsid protein corresponding to amino acid positions 346, 501, 505, and 706 of SEQ ID NO: 14 is not the wild type amino acid. In certain embodiments, the instant disclosure provides a recombinant AAV capsid protein, wherein the amino acid in the capsid protein corresponding to amino acid positions 346, 501, 705, and 706 of SEQ ID NO: 14 is not the wild type amino acid. In certain embodiments, the instant disclosure provides a recombinant AAV capsid protein, wherein the amino acid in the capsid protein corresponding to amino acid positions 346, 505, 705, and 706 of SEQ ID NO: 14 is not the wild type amino acid.

In certain embodiments, the instant disclosure provides a recombinant AAV capsid protein, wherein the amino acid in the capsid protein corresponding to amino acid positions 346, 501, 505, 705, and 706 of SEQ ID NO: 14 is not the wild type amino acid.

In another aspect, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the amino acid in the capsid protein corresponding to amino acid position 501 of SEQ ID NO: 14 is leucine, threonine, tyrosine, or tryptophan.

In certain embodiments, the instant disclosure provides a recombinant adeno-associated virus (AAV) capsid protein, wherein: the amino acid in the capsid protein corresponding to amino acid position 346 of SEQ ID NO: 14 is alanine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is glycine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is leucine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is threonine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is tryptophan, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is tyrosine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 705 of SEQ ID NO: 14 is leucine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; and the amino acid in the capsid protein corresponding to amino acid position 501 of SEQ ID NO: 14 is isoleucine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 501 of SEQ ID NO: 14 is isoleucine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the recombinant AAV capsid protein is a variant of a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein.

In certain embodiments, the instant disclosure provides a recombinant adeno-associated virus (AAV) capsid protein, wherein the amino acid in the capsid protein corresponding to amino acid positions 346, 501, 505, 705, and/or 706 of SEQ ID NO: 14 is not the wild type amino acid (i.e., not the amino acid that occurs in nature), and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 1-30. In certain embodiments, the instant disclosure provides a recombinant adeno-associated virus (AAV) capsid protein, wherein the amino acid in the capsid protein corresponding to amino acid positions 346, 501, 505, 705, and/or 706 of SEQ ID NO: 14 is not the wild type amino acid (i.e., not the amino acid that occurs in nature), and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid position 346 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid position 346 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid position 501 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid position 501 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid position 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid position 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid position 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid position 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid position 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid position 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346 and 501 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346 and 501 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346 and 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346 and 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346 and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346 and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 501 and 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 501 and 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 501 and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 501 and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 501 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 501 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 505 and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 505 and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 505 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 505 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 705 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 705 and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 501, and 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 501, and 505 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 501, and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 501, and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 501, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 501, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 505, and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 505, and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 505, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 505, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 501, 505, and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 501, 505, and 705 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 501, 505, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 501, 505, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 501, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 501, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 505, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 505, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 501, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 501, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 501, 505, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 501, 505, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 505, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 505, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant AAV capsid protein, wherein the amino acid in the capsid protein corresponding to amino acid positions 346, 501, 505, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant AAV capsid protein, wherein the amino acid in the capsid protein corresponding to amino acid positions 346, 501, 505, 705, and 706 of SEQ ID NO: 14 is not the wild type amino acid, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and/or the amino acid in the capsid protein corresponding to amino acid position 706 of SEQ ID NO: 14 is cysteine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and/or the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the amino acid in the capsid protein corresponding to amino acid position 501 of SEQ ID NO: 14 is leucine, threonine, tyrosine, or tryptophan.

In certain embodiments, the instant disclosure provides a recombinant adeno-associated virus (AAV) capsid protein, wherein: the amino acid in the capsid protein corresponding to amino acid position 346 of SEQ ID NO: 14 is alanine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is glycine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is glycine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is leucine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is leucine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is threonine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is threonine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is tryptophan; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is tryptophan, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is tyrosine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is tyrosine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 505 of SEQ ID NO: 14 is arginine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 705 of SEQ ID NO: 14 is leucine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 705 of SEQ ID NO: 14 is leucine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 706 of SEQ ID NO: 14 is cysteine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; and the amino acid in the capsid protein corresponding to amino acid position 501 of SEQ ID NO: 14 is isoleucine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; and the amino acid in the capsid protein corresponding to amino acid position 501 of SEQ ID NO: 14 is isoleucine; and the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; and the amino acid in the capsid protein corresponding to amino acid position 505 of SEQ ID NO: 14 is arginine; and the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; and the amino acid in the capsid protein corresponding to amino acid position 706 of SEQ ID NO: 14 is cysteine; and the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; and the amino acid in the capsid protein corresponding to amino acid position 505 of SEQ ID NO: 14 is arginine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; and the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; and the amino acid in the capsid protein corresponding to amino acid position 706 of SEQ ID NO: 14 is cysteine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 505 of SEQ ID NO: 14 is arginine; and the amino acid in the capsid protein corresponding to amino acid position 706 of SEQ ID NO: 14 is cysteine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 501 of SEQ ID NO: 14 is isoleucine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 501 of SEQ ID NO: 14 is isoleucine, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; and the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 505 of SEQ ID NO: 14 is arginine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and the amino acid in the capsid protein corresponding to amino acid position 706 of SEQ ID NO: 14 is cysteine; wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15; and wherein the recombinant AAV capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NOs: 1-30. In certain embodiments, the instant disclosure provides a recombinant adeno-associated virus (AAV) capsid protein, wherein: the amino acid in the capsid protein corresponding to amino acid position 346 of SEQ ID NO: 14 is alanine or glycine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; the amino acid in the capsid protein corresponding to amino acid position 705 of SEQ ID NO: 14 is leucine; and the amino acid in the capsid protein corresponding to amino acid position 706 of SEQ ID NO: 14 is cysteine, and wherein the capsid protein comprises an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to any one of the amino acid sequences of SEQ ID NOs: 31-411 and 453-1304.

In certain embodiments, the instant disclosure provides a recombinant adeno-associated virus (AAV) capsid protein comprising any one of the amino acid sequences of SEQ ID NOs: 31-411 and 453-1304.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-736 of any one of SEQ ID NOs: 31-197, 220-234, 250-264, 280-306, and 397-411, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-736 of any one of SEQ ID NOs: 31-197, 220-234, 250-264, 280-306, and 397-411, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-736 of any one of SEQ ID NOs: 31-197, 220-234, 250-264, 280-306, and 397-411, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-736 of any one of SEQ ID NOs: 31-197, 220-234, 250-264, 280-306, and 397-411, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-736 of any one of SEQ ID NOs: 31-197, 220-234, 250-264, 280-306, and 397-411, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-736 of any one of SEQ ID NOs: 31-197, 220-234, 250-264, 280-306, and 397-411, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 197-736 of any one of SEQ ID NOs: 198-204, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 142-736 of any one of SEQ ID NOs: 198-204, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-736 of any one of SEQ ID NOs: 198-204, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 197-736 of any one of SEQ ID NOs: 198-204, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 142-736 of any one of SEQ ID NOs: 198-204, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-736 of any one of SEQ ID NOs: 198-204, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-735 of any one of SEQ ID NOs: 205-219, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-735 of any one of SEQ ID NOs: 205-219, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-735 of any one of SEQ ID NOs: 205-219, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-735 of any one of SEQ ID NOs: 205-219, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-735 of any one of SEQ ID NOs: 205-219, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-735 of any one of SEQ ID NOs: 205-219, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 193-724 of any one of SEQ ID NOs: 235-249, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 137-724 of any one of SEQ ID NOs: 235-249, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-724 of any one of SEQ ID NOs: 235-249, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 193-724 of any one of SEQ ID NOs: 235-249, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 137-724 of any one of SEQ ID NOs: 235-249, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-724 of any one of SEQ ID NOs: 235-249, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 204-738 of any one of SEQ ID NOs: 265-279, and 307-321, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-738 of any one of SEQ ID NOs: 265-279, and 307-321, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-738 of any one of SEQ ID NOs: 265-279, and 307-321, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 204-738 of any one of SEQ ID NOs: 265-279, and 307-321, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-738 of any one of SEQ ID NOs: 265-279, and 307-321, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-738 of any one of SEQ ID NOs: 265-279, and 307-321, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-737 of any one of SEQ ID NOs: 337-366, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-737 of any one of SEQ ID NOs: 337-366, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-737 of any one of SEQ ID NOs: 337-366, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-737 of any one of SEQ ID NOs: 337-366, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-737 of any one of SEQ ID NOs: 337-366, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-737 of any one of SEQ ID NOs: 337-366, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-738 of any one of SEQ ID NOs: 367-381, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-738 of any one of SEQ ID NOs: 367-381, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-738 of any one of SEQ ID NOs: 367-381, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 203-738 of any one of SEQ ID NOs: 367-381, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-738 of any one of SEQ ID NOs: 367-381, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-738 of any one of SEQ ID NOs: 367-381, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 202-729 of any one of SEQ ID NOs: 322-336, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 136-729 of any one of SEQ ID NOs: 322-336, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-729 of any one of SEQ ID NOs: 322-336, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 202-729 of any one of SEQ ID NOs: 322-336, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 136-729 of any one of SEQ ID NOs: 322-336, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-729 of any one of SEQ ID NOs: 322-336, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 204-738 of any one of SEQ ID NOs: 382-396, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-738 of any one of SEQ ID NOs: 382-396, wherein: the amino acid in the capsid protein corresponding to amino acid positions 346, 501, 505, and/or 706 of SEQ ID NO: 14 is not the wild type amino acid. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-738 of any one of SEQ ID NOs: 382-396, wherein: the amino acid in the capsid protein corresponding to amino acid positions 346, 501, 505, and/or 706 of SEQ ID NO: 14 is not the wild type amino acid.

In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 204-738 of any one of SEQ ID NOs: 382-396, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 138-738 of any one of SEQ ID NOs: 382-396, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine. In certain embodiments, the instant disclosure provides a recombinant adeno-associated virus (AAV) capsid protein comprising an amino acid sequence that is at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to amino acids 1-738 of any one of SEQ ID NOs: 382-396, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine.

### III. POLYNUCLEOTIDES, VECTORS, AND METHODS OF PRODUCING AAV CAPSIDS

In another aspect, the instant disclosure provides an isolated polynucleotide encoding a recombinant AAV capsid protein disclosed herein.

In certain embodiments, the instant disclosure provides a polynucleotide encoding a recombinant adeno-associated virus (AAV) capsid protein, wherein the amino acid in the capsid protein corresponding to amino acid positions 346, 501, 505, and/or 706 of SEQ ID NO: 14 is not the wild type amino acid. In certain embodiments, the instant disclosure provides a polynucleotide encoding a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In certain embodiments, the polynucleotide is optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of the encoded capsid protein relative to the expression of the capsid encoded by polynucleotides that have not been optimized.

In another aspect the instant disclosure provides a vector comprising a polynucleotide disclosed herein. Suitable vectors, include, without limitation, plasmids, viruses, cosmids, artificial chromosomes, linear DNA, and mRNA. In certain embodiments, the vector is a plasmid or a viral vector. In certain embodiments, the vector is a retrovirus vector, a herpes virus vector, a baculovirus vector, or an adenovirus vector. In certain embodiments, the vector is an expression vector.

Vectors (e.g., expression vectors) can be introduced into cells (using any techniques known in the art) for propagation of the vector and/or for expression of an AAV capsid protein encoded by the vector. Accordingly, in another aspect, the instant disclosure provides a recombinant cell comprising a polynucleotide or a vector (e.g., an expression vector) disclosed herein. And further, in another aspect, the instant disclosure provides a method of producing an AAV capsid protein, the method comprising culturing the recombinant cell under conditions whereby the polynucleotide is expressed and the capsid is produced.

Suitable vectors, include, without limitation, plasmids, minimal vectors (e.g., minicircles, Nanoplasmids™, doggybones, MIDGE vectors, and the like), viruses, cosmids, artificial chromosomes, linear DNA, and mRNA. In certain embodiments, the first nucleic acid vector and/or the second nucleic acid vector is a DNA plasmid or a DNA minimal vector. Any DNA plasmid or DNA minimal vector that can accommodate the necessary vector elements can be used for the first nucleic acid vector and the second nucleic acid vector. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™, minimalistic immunologically defined gene expression (MIDGE) vectors, and others known to those of skill in the art. DNA minimal vectors and their methods of production are described in, e.g., U.S. Patent Application Nos. 20100233814; 20120282283; 20130216562; 20150218565; 20150218586; 20160008488; 20160215296; 20160355827; 20190185924; 20200277624; and 20210010021, all of which are herein incorporated by reference in their entireties.

A variety of host cells and expression vector systems can be utilized to express the capsid proteins described herein. Such expression systems represent vehicles by which the coding sequences of interest can be produced and subsequently purified, but also represent cells which can, when transformed or transfected with the appropriate nucleotide coding sequences, express a capsid protein described herein in situ. These include but are not limited to microorganisms such as bacteria (e.g., E. coli and B. subtilis) transformed with, e.g., recombinant bacteriophage DNA, plasmid DNA, or cosmid DNA expression vectors containing capsid protein coding sequences; yeast (e.g., Saccharomyces Pichia) transformed with, e.g., recombinant yeast expression vectors containing capsid protein coding sequences; insect cell systems infected with, e.g., recombinant virus expression vectors (e.g., baculovirus) containing capsid protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing capsid protein coding sequences; or mammalian cell systems (e.g., COS (e.g., COS1 or COS), CHO, BHK, MDCK, HEK 293, NSO, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, cells for expressing the capsid proteins described herein are human cells, e.g., human cell lines. In certain embodiments, a mammalian expression vector is pOptiVEC™ or pcDNA3.3. In certain embodiments, bacterial cells such as Escherichia coli, or eukaryotic cells (e.g., mammalian cells), are used for the expression of a capsid protein. For example, mammalian cells such as CHO or HEK293 cells, in conjunction with a vector such as the major intermediate early gene promoter element from human cytomegalovirus is an effective expression system for capsid proteins disclosed herein.

In bacterial systems, a number of expression vectors can be advantageously selected depending upon the use intended for the capsid protein being expressed. For example, when a large quantity of a capsid protein is to be produced, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the capsid protein coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The capsid protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the capsid protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the capsid protein molecule in infected hosts (see, e.g., Logan J & Shenk T (1984) PNAS 81(12): 3655-9, which is herein incorporated by reference in its entirety). Specific initiation signals can also be required for efficient translation of inserted capsid protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al. (1987) Methods Enzymol. 153: 516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7O3O, COS (e.g., COS1 or COS), PER.C6, VERO, HEK-293T, HepG2, SP210, R1.1, B-W, L-M, BSC1, BSC40, YB/20, BMT10, and HsS78Bst cells.

For long-term, high-yield production of recombinant proteins, stable expression cells can be generated. For example, cell lines which stably express a capsid protein described herein can be engineered.

In certain embodiments, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a capsid protein described herein or a fragment thereof.

A number of selection systems can be used, including but not limited to the herpes simplex virus thymidine kinase (Wigler M et al. (1977) Cell 11(1): 223-32); hypoxanthineguanine phosphoribosyltransferase (Szybalska E H & Szybalski W (1962) PNAS 48(12): 2026-2034); and adenine phosphoribosyltransferase (Lowy I et al. (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al. (1980) PNAS 77(6): 3567-70; O'Hare K et al. (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al. (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al. (eds.), Current Protocols in Molecular Biology, John Wiley & Sons, N Y (1993); Kriegler M, Gene Transfer and Expression, A Laboratory Manual, Stockton Press, N Y (1990); and in Chapters 12 and 13, Dracopoli N C et al. (eds.), Current Protocols in Human Genetics, John Wiley & Sons, N Y (1994); Colbere-Garapin F et al. (1981) J Mol Biol 150: 1-14, all of which are herein incorporated by reference in their entireties.

### IV. AAV COMPOSITIONS

In another aspect, the instant disclosure provides an rAAV composition comprising a capsid comprising a recombinant AAV capsid protein disclosed herein.

In certain embodiments, the instant disclosure provides an rAAV composition comprising a capsid comprising a recombinant adeno-associated virus (AAV) capsid protein, wherein the amino acid in the capsid protein corresponding to amino acid positions 346, 501, 505, and/or 706 of SEQ ID NO: 14 is not the wild type amino acid. In certain embodiments, the instant disclosure provides an rAAV composition comprising a capsid comprising a recombinant adeno-associated virus (AAV) capsid protein, wherein: the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, and wherein the capsid protein does not comprise the amino acid sequence of amino acids 203-736 of SEQ ID NOs: 11-15.

In another aspect, the instant disclosure provides an rAAV comprising a capsid comprising one or more recombinant capsid proteins disclosed herein; and an rAAV genome. The rAAVs disclosed herein generally comprise a recombinant genome (e.g., an rAAV genome) packaged within the capsid. The rAAV genome can be of any type that is capable of being packages within an AAV capsid disclosed herein. For example, in certain embodiments, the rAAV genome is a single-stranded DNA genome. In certain embodiments, the rAAV genome is a self-complementary genome, for example as described in U.S. Pat. No. 7,790,154, which is hereby incorporated by reference in its entirety.

In certain embodiments, the rAAV genome comprises a transgene. In certain embodiments, the transgene encodes a polypeptide. In certain embodiments, the transgene encodes a therapeutic protein. In certain embodiments, the transgene encodes an antibody or a fragment thereof (e.g., a Fab, scFv, or full-length antibody). In certain embodiments, the transgene encodes an scFv, nanobody, or VHH. In certain embodiments, the transgene encodes a non-coding RNA.

In certain embodiments, the rAAV genome comprises a transgene. In certain embodiments the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-10, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2, and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrofin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes a nuclease. Suitable nucleases include, without limitation, zinc fingers nucleases (ZFN) (see, e.g., Porteus, and Baltimore (2003) Science 300: 763; Miller et al. (2007) Nat. Biotechnol. 25: 778-785; Sander et al. (2011) Nature Methods 8: 67-69; and Wood et al. (2011) Science 333: 307, each of which is hereby incorporated by reference in its entirety); transcription activator-like effectors nucleases (TALEN) (see, e.g., Wood et al. (2011) Science 333: 307; Boch et al. (2009) Science 326: 1509-1512; Moscou and Bogdanove (2009) Science 326: 1501; Christian et al. (2010) Genetics 186: 757-761; Miller et al. (2011) Nat. Biotechnol. 29: 143-148; Zhang et al. (2011) Nat. Biotechnol. 29: 149-153; and Reyon et al. (2012) Nat. Biotechnol. 30(5): 460-465, each of which is hereby incorporated by reference in its entirety); homing endonucleases; meganucleases (see, e.g., U.S. Patent Publication No. US 2014/0121115, which is hereby incorporated by reference in its entirety); and RNA-guided nucleases (see, e.g., Makarova et al. (2018) The CRISPR Journal 1(5): 325-336; and Adli (2018) Nat. Communications 9: 1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type I-D (Cas10d), type I-E (Cse1, Cse2), type I-F (Csy1, Csy2, Csy3), type I-U (GSU0054), type III (Cas10), type III-A (Csm2), type III-B (Cmr5), type III-C(Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV genome comprises a TRE operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements, including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441); CMV early enhancer/chicken β-actin (CBA) promoter/rabbit β-globin intron (CAG) (Miyazaki et al. (1989) Gene 79(2): 269-277); CB^{SB} (Jacobson et al. (2006) Molecular Therapy 13(6): 1074-1084); human elongation factor 1α promoter (EF1α) (Kim et al. (1990) Gene 91 (2): 217-223); human phosphoglycerate kinase promoter (PGK) (Singer-Sam et al. (1984) Gene 32(3): 409-417); mitochondrial heavy-strand promoter (Lodeiro et al. (2012) PNAS 109(17): 6513-6518); and ubiquitin promoter (Wulff et al. (1990) FEBS Letters 261: 101-105). In certain embodiments, the TRE comprises a cytomegalovirus (CMV) promoter/enhancer (e.g., comprising a nucleotide sequence at least 90%, 91%, 92%, 93%, 94%, 95%, 96%, 97%, 98%, 99%, or 100% identical to SEQ ID NO: 412 or 413), an SV40 promoter, a chicken beta actin (CBA) promoter (e.g., comprising a nucleotide sequence at least 90%, 91%, 92%, 93%, 94%, 95%, 96%, 97%, 98%, 99%, or 100% identical to SEQ ID NO: 414 or 415), an smCBA promoter (e.g., comprising a nucleotide sequence at least 90%, 91%, 92%, 93%, 94%, 95%, 96%, 97%, 98%, 99%, or 100% identical to SEQ ID NO: 416), a human elongation factor 1 alpha (EF1α) promoter (e.g., comprising a nucleotide sequence at least 90%, 91%, 92%, 93%, 94%, 95%, 96%, 97%, 98%, 99%, or 100% identical to SEQ ID NO: 417), a minute virus of mouse (MVM) intron which comprises transcription factor binding sites (e.g., comprising a nucleotide sequence at least 90%, 91%, 92%, 93%, 94%, 95%, 96%, 97%, 98%, 99%, or 100% identical to SEQ ID NO: 418 or 419), a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element (e.g., comprising a nucleotide sequence at least 90%, 91%, 92%, 93%, 94%, 95%, 96%, 97%, 98%, 99%, or 100% identical to SEQ ID NO: 420 or 421), a human calmodulin 1 (CALM1) promoter (e.g., comprising a nucleotide sequence at least 90%, 91%, 92%, 93%, 94%, 95%, 96%, 97%, 98%, 99%, or 100% identical to SEQ ID NO: 422), a human ApoE/C-I hepatic control region (HCR1) (e.g., comprising a nucleotide sequence at least 90%, 91%, 92%, 93%, 94%, 95%, 96%, 97%, 98%, 99%, or 100% identical to SEQ ID NO: 423), a human al-antitrypsin (hAAT) promoter (e.g., comprising a nucleotide sequence at least 90%, 91%, 92%, 93%, 94%, 95%, 96%, 97%, 98%, 99%, or 100% identical to SEQ ID NO: 424, 425, or 426), an extended HCR1 (e.g., comprising a nucleotide sequence at least 90%, 91%, 92%, 93%, 94%, 95%, 96%, 97%, 98%, 99%, or 100% identical to SEQ ID NO: 431), a HS-CRM8 element of an hAAT promoter (e.g., comprising a nucleotide sequence at least 90%, 91%, 92%, 93%, 94%, 95%, 96%, 97%, 98%, 99%, or 100% identical to SEQ ID NO: 432), a human transthyretin (TTR) promoter (e.g., comprising a nucleotide sequence at least 90%, 91%, 92%, 93%, 94%, 95%, 96%, 97%, 98%, 99%, or 100% identical to SEQ ID NO: 427), and/or a human Methyl-CpG Binding Protein 2 (MeCP2) promoter. Any of the TREs described herein can be combined in any order to drive efficient transcription. For example, a transfer genome may comprise a TRE comprising a CMV enhancer, a CBA promoter, and the splice acceptor from exon 3 of the rabbit beta-globin gene, collectively called a CAG promoter (e.g., comprising a nucleotide sequence at least 90%, 91%, 92%, 93%, 94%, 95%, 96%, 97%, 98%, 99%, or 100% identical to SEQ ID NO: 428). For example, a transfer genome may comprise a TRE comprising a hybrid of CMV enhancer and CBA promoter followed by a splice donor and splice acceptor, collectively called a CASI promoter region (e.g., comprising a nucleotide sequence at least 90%, 91%, 92%, 93%, 94%, 95%, 96%, 97%, 98%, 99%, or 100% identical to SEQ ID NO: 429). For example, a transfer genome may comprise a TRE comprising a HCR1 and hAAT promoter (also referred to as an LP1 promoter, e.g., comprising a nucleotide sequence at least 90%, 91%, 92%, 93%, 94%, 95%, 96%, 97%, 98%, 99%, or 100% identical to SEQ ID NO: 430).

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the rAAV genome comprises an editing genome. Editing genomes can be used to edit the genome of a cell by homologous recombination of the editing genome with a genomic region surrounding a target locus in the cell. In certain embodiments, the editing genome is designed to correct a genetic defect in a gene by homologous recombination. Suitable target genes for editing using an editing genome include, without limitation, phenylalanine hydroxylase (PAH), cystic fibrosis conductance transmembrane regulator (CFTR), beta hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y).

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### V. METHODS OF USE

In another aspect, the instant disclosure provides methods for transducing a cell. The methods generally comprise contacting the cell with an rAAV disclosed herein under conditions whereby the cell is transduced.

The rAAV disclosed herein can comprise a transgene under the control of a TRE. Accordingly, in certain embodiments, the instant disclosure provides methods for expressing a transgene in a cell, the method generally comprising contacting the cell with such an rAAV under conditions whereby the cell is transduced and the transgene is expressed. The transgene can encode a polypeptide and/or an RNA molecule, as described herein. Accordingly, in certain embodiments, the instant disclosure provides methods for producing a polypeptide and/or an RNA molecule in a cell, the method generally comprising contacting the cell with such an rAAV under conditions whereby the cell is transduced and the polypeptide and/or an RNA molecule is produced.

The rAAV disclosed herein can comprise an editing genome. rAAV comprising editing genomes can be used to edit the genome of a cell by homologous recombination of the editing genome with a homologous target locus in the cell. Accordingly, in certain embodiments, the instant disclosure provides a method for editing a target locus in a genome of a cell, the method generally comprising contacting the cell with such an rAAV under conditions whereby the cell is transduced and the target locus is edited.

The rAAV disclosed herein can be used to transduce cells in vitro, in vivo, and ex vivo.

The rAAV disclosed herein can be administered to a subject (e.g., a human subject) by all routes suitable for an rAAV, including, without limitation, intravenously, intraperitoneally, subcutaneously, intramuscularly, intrathecally, or intradermally.

In another aspect, the invention provides an rAAV as disclosed herein for use in medicine. In another aspect, the invention provides an rAAV as disclosed herein for use as therapy. In another aspect, the invention provides an rAAV as disclosed herein for use as a medicament.

### VI. ADENO-ASSOCIATED VIRUS PACKAGING SYSTEMS

In another aspect, the instant disclosure provides packaging systems for recombinant preparation of a recombinant adeno-associated virus (rAAV) disclosed herein. Such packaging systems generally comprise: first nucleotide encoding one or more AAV Rep proteins; a second nucleotide encoding a capsid protein of any of the AAVs as disclosed herein; and a third nucleotide sequence comprising any of the rAAV genome sequences as disclosed herein, wherein the packaging system is operative in a cell for enclosing the transfer genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.). In certain embodiments of the packaging system, the packaging system further comprises a fourth nucleotide sequence comprising one or more helper virus genes. In certain embodiments, the fourth nucleotide sequence comprises adenoviral E2, E4, and VA genes. In certain embodiments of the packaging system, the packaging system further comprises a third vector (e.g., a helper virus vector), comprising the fourth nucleotide sequence. The third vector may be an independent third vector, integral with the first vector, or integral with the second vector.

In certain embodiments, the packaging system comprises a first vector comprising the first nucleotide sequence encoding one or more AAV Rep proteins, the second nucleotide sequence encoding a capsid protein of any of the AAVs as disclosed herein, and the third nucleotide sequence comprising any of the rAAV genome sequences as disclosed herein, wherein the packaging system is operative in a cell for enclosing the transfer genome in the capsid to form the AAV. In certain embodiments of the packaging system, the packaging system further comprises a fourth nucleotide sequence comprising one or more helper virus genes. In certain embodiments, the fourth nucleotide sequence comprises adenoviral E2, E4, and VA genes. In certain embodiments of the packaging system, the packaging system further comprises a second vector (e.g., a helper virus vector), comprising the fourth nucleotide sequence. The second vector may be an independent second vector, integral with the first vector.

Any AAV Rep protein can be employed in the packaging systems disclosed herein. In certain embodiments of the packaging system, the Rep nucleotide sequence encodes an AAV2 Rep protein. Suitable AAV2 Rep proteins may include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO: 433, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO: 433.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4, and VA. In certain embodiments of the packaging system, where the helper virus is adenovirus, the adenovirus genome comprises one or more adenovirus RNA genes selected from the group consisting of E2, E4, and VA. In certain embodiments of the packaging system, where the helper virus is HSV, the HSV genome comprises one or more of HSV genes selected from the group consisting of UL5/8/52, ICPO, ICP4, ICP22, and UL30/UL42.

In certain embodiments of the packaging system, the vectors (e.g., first, second, and/or third vectors) are contained within one or more plasmids.

In certain embodiments of the packaging system, the first, second, and/or third vectors are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g., with one or more recombinant helper viruses, such as a adenovirus, poxvirus (such as vaccinia virus), herpes virus (including HSV, cytomegalovirus, or baculovirus, containing a first, and a second, and optionally a third vector as described herein)), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more capsid proteins as described herein, and with a transfer genome as described herein being delivered in the form of a plasmid or a recombinant helper virus).

Accordingly, the instant disclosure provides a packaging system for preparation of an rAAV, wherein the packaging system comprises: a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes (e.g., adenoviral E2, E4, and VA genes).

### VII. EXAMPLES

The following examples are offered by way of illustration, and not by way of limitation.

### Materials and Methods

### Culturing of Cell Lines.

CHO glycan mutants, Pro5, Lec2, Lec1, and Lec8 cells, were cultured in minimum essential medium alpha (MEM a) with nucleosides (ThermoFisher Scientific, Waltham, MA) and supplemented with 10% FBS (ThermoFisher Scientific, Waltham, MA) and 1% Penicillin-Streptomycin (ThermoFisher Scientific, Waltham, MA).

rAAV Production, Purification, and Titration.

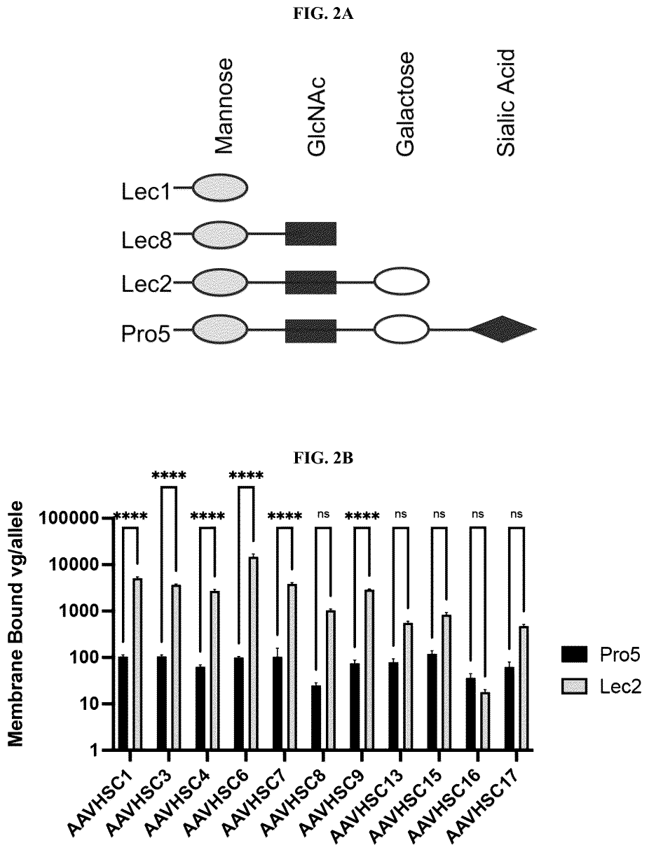

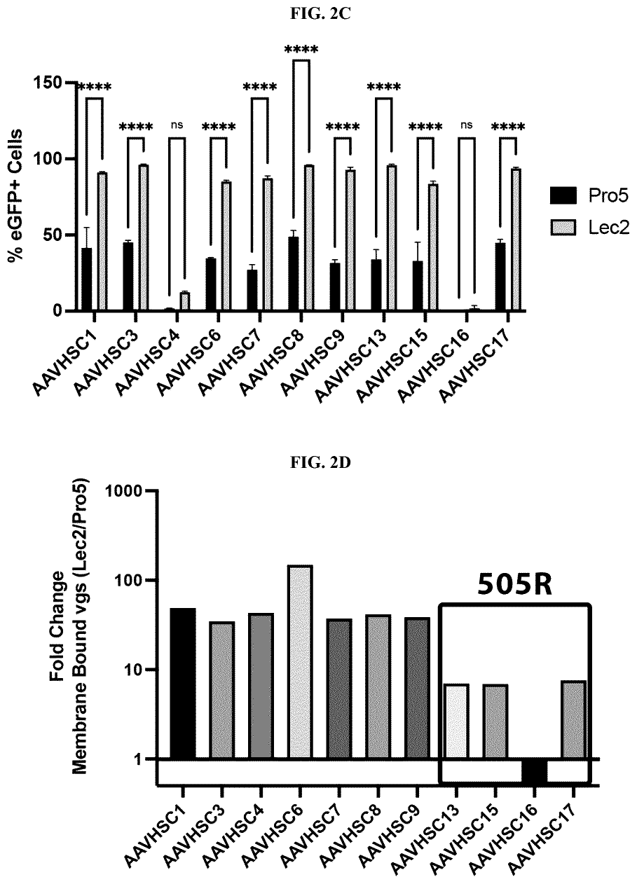

For vectors used in vitro studies, recombinant AAVs were packaged in different AAV and AAVHSC capsids with self-complementary CBA-eGFP (abbreviated hereafter as scCBA.eGFP), containing the chicken beta actin promoter, CMV enhancer, eGFP, and simian virus 40 (SV40) late polyA. Vectors in FIGS. 2B-2D were packaged and prepared by triple-plasmid transient transfection in human embryonic kidney (HEK)293 cells using calcium phosphate and purified through two rounds of cesium chloride (CsCl) density gradient ultracentrifugation (SABTech, Inc., Philadelphia, PA). All AAV vectors were formulated in PBS-based buffer. Vector titers were determined by quantitative PCR (qPCR) using primers and probes specific for eGFP.

All remaining vectors used in in vitro and in vivo studies were manufactured by triple transfection of HEK293 cells with a plasmid containing the payload of interest containing AAV2 ITRs, a second plasmid containing the capsid sequence and AAV2 Rep gene, and a third plasmid containing adenovirus helper genes. At 72 hours post-transfection, cells were separated from supernatant by centrifugation and lysed for 1 hour in a buffer containing Tris-HCl, sodium-chloride (NaCl), Triton X-100, magnesium chloride (MgCl2), and benzonase. Cell debris was clarified by centrifugation and purified using an AAV9 affinity resin. Vectors were enriched for full capsids by cesium ultracentrifugation and buffer exchanged and formulated in DPBS-based buffer. Vectors were titrated by quantitative PCR using primers targeting the SV40 region. All vectors were analyzed by silver and Coomassie Blue-stained sodium dodecyl-sulfate polyacrylamide gel electrophoresis (SDS-PAGE) for VP1, 2, and 3 ratios, endotoxin (<10 EU/mL) and capsid content by enzyme-linked immunoassay (ELISA). All AAVHSC and altered AAVHSC capsids were able to yield acceptable titers when produced using the same process (Table 1).

TABLE 1
Titers of AAVHSC and AAVHSC Variants

| AAVHSC Serotype | Titer (VG/mL) |
|---|---|
| AAVHSC7 | 1.25E+13 |
| AAVHSC7 F501I | 4.21E+13 |
| AAVHSC15 | 3.64E+13* |
| AAVHSC15 F501I | 4.56E+13 |
| AAVHSC15 Y706C | 1.75E+13 |
| AAVHSC16 | 3.33E+13 |
| AAVHSC16 I501F | 2.83E+13 |
| AAVHSC16 Y706C | 4.56E+13 |
| AAVHSC16 T346A | 3.17E+13 |
| AAVHSC16 I501L | 2.00E+13 |
| AAVHSC16 I501T | 1.30E+13 |
| AAVHSC16 I501V | 1.20E+13 |
| AAVHSC16 I501W | 1.32E+13 |
| AAVHSC16 I501YA | 9.16E+12 |
| AAVHSC16 I501YB | 1.44E+13 |
| AAVHSC7 G505R | 4.56E+13 |
| AAVHSC8 | 1.03E+13 |
| AAVHSC8 G505R | 4.39E+13 |

*average of three separate productions.

### Mutagenesis of AAVHSCs.

Mutagenic oligonucleotides were synthesized to induce single nucleotide changes (Integrated DNA Technologies, Coralville, IA). Mutations were introduced onto the wt AAVHSC7, AAVHSC15, and AAVHSC16 capsids using the QuikChange Lightning Site-Directed Mutagenesis Kit (Agilent Technologies, Santa Clara, CA) according to the manufacturer's specifications. The mutagenized plasmid was then transformed into DH5α Turbo Competent Escherichia coli (New England Biolabs, Ipswich, MA) and colonies screened by DNA sequencing (Eton Biosciences, Charlestown, MA).

### CHO Cell Surface Binding Assay.

Cells were counted and resuspended in growth media at a density of 1-2×10⁶ cells/mL and plated in pre-chilled 96-well tissue culture plates and then incubated on ice for 15 minutes prior to transduction. Cells were then transduced on ice by adding the scCBA.eGFP vectors directly to cell culture media at the indicated MOL. 1 hour following transduction, cells were washed 3 times in 4° C. PBS. After the final wash, half of the cells were resuspended in 100 μL PBS for subsequent DNA extraction using the In-Plate Cell Lysis and DNA Extraction Method. The remaining half of the cells were resuspended in media and cultured at 37° C. for 24 hours.

### Transduction Assay in Primary Human Hepatocytes.

Frozen primary human hepatocytes were plated at 2×10⁵ cells/well in a collagen coated 48-well plate. At 6 hours following plating, culture medium was replaced with fresh medium and cells were transduced with scCBA.eGFP vectors at increasing MOIs of 1.5×10⁵, 3×10⁵, and 6×10⁵. After 48 hours post-transduction, culture medium was replaced. Fluorescent microscopy was used to detect eGFP expression at day four post-transduction.

### Flow Cytometric Analysis.

In vitro eGFP expression was measured at 24 hours post AAV scCBA.eGFP transduction by flow cytometric analysis on either the SONY SH800 (Sony Biotechnology) or the CytoFLEX S (Beckman Coulter Inc., Brea, California). Briefly, cells were washed once with PBS and adherent CHO cells were dissociated using TrypLE Reagent (ThermoFisher Scientific, Waltham, MA). Cells were then stained to determine viability using LIVE/DEAD™ Fixable Violet Dead Cell Stain (ThermoFisher Scientific, Waltham, MA) for 10 minutes at room temperature in PBS. Cells were washed and then resuspended in FACS buffer (2% fetal bovine serum in PBS) or Cell Staining buffer (BioLegend, San Diego, CA). A minimum of 1×10⁴ cells were collected for each sample. The total number of cells and level of terminally exposed cell membrane galactose was determined using Lectin Peanut Agglutinin (PNA) (Item No. L32460, ThermoFisher Scientific, Waltham, MA) from Arachis hypogaea (peanut), conjugated with Alexa Fluor 647. Cells were stained using a 1:1000 dilution, incubated for 1 hour and then washed twice with PBS prior to flow cytometry analysis.

DNA Isolation from Cells.

Direct quantification of DNA from lysed cells was performed through an in-plate lysis protocol. Briefly, to each 100 μL cell suspension sample, 85 μL of extraction solution (0.25% deoxycholate, 0.45% Tween/HEPES solution (25% Tween-20, 0.5M HEPES), 1 mM Tris HCl, 1 mM EDTA, 0.1% sodium dodecyl sulfate, 0.3 mg/mL Proteinase K) (ThermoFisher Scientific, Waltham, MA) was added. Samples were then mixed by pipetting and sealed with an adhesive plate film. The plate was then transferred to a thermocycler and incubated at 37° C. for 1 hour, 55° C. for 2 hours, and 95° C. for 30 minutes (to inactivate the Proteinase K). For subsequent qPCR analysis, 10 μL of digestion per sample was diluted in 100 μL of Ultrapure water (ThermoFisher Scientific, Waltham, MA).

### Quantitation of VGs in Transduced Cells.

The ratio of AAV vgs per cell or allele was determined by quantitative real-time PCR (qPCR) of DNA isolated from cells. Analysis was performed on the QuantStudio 3 Real-Time PCR System (Applied Biosystems, ThermoFisher Scientific, Waltham, MA). VG detection was using TaqMan™ Universal PCR Master Mix (ThermoFisher Scientific, Waltham, MA) and the primers and probes set forth in Table 2 (Integrated DNA Technologies, Coralville, IA):

TABLE 2
Primers and Probes for ddPCR

| | | | SEQ |
|---|---|---|---|
| | | | ID |
| | | Sequence | NO: |
| eGFP | Forward | 5′-CTGCTGCCCGACAACCA-3′ | 434 |
| #1 | Reverse | 5′-GACCATGTGATCGCGCTTCT-3′ | 435 |
| | Probe | 5′-TACCTGAGCACCCAGTCCGCCCT-3′ | 436 |
| Human | Forward | 5′-TGAAGGTGGAGGACATTCCTCTA-3′ | 437 |
| ApoB | Reverse | 5′-CTGGAATTGCGATTTCTGGTAA-3′ | 438 |
| | Probe | 5′-CGAGAATCACCCTGCCAGACTTCCGT-3′ | 439 |
| CHO | Forward | 5′-CCATGTACGTAGCCATTCAGG-3′ | 440 |
| Actin | Reverse | 5′-CATGAGGGAGAGCGTAGCC-3′ | 441 |
| Beta | Probe | 5′-TGTCCCTGTATGCCTCTGGTCGTA-3′ | 442 |

### Animal Procedures for Mice.

Housing and all experimental procedures were in accordance with the Institutional Animal Care and Use Committee (IACUC). B6(Cg)-Tyrc-2J/J (B6 albino) mice (obtained from Jackson Laboratories, Bar Harbor, ME, USA) and CD1 mice (obtained from Charles River Laboratory, Wilmington, MA), were maintained in the animal facility under standard laboratory conditions (temperature 20-24° C., relative humidity 50-60%, 12 hour light/12 hour dark cycle). Mice were supplied with standard chow (Item No. PicoLab 5058, LabDiet, St. Louis, MO) and sterile water ad libitum. Male B6 albino mice at 7-weeks of age and male and female CD1 mice at 4-weeks of age were administered vectors or vehicle by IV injection using a 1 mL syringe with 28-gauge needle. Imaging of dosed B6 albino mice was performed at 6 weeks post-injection, XenoLight D-luciferin-K+ Salt Bioluminescent Substrate (Item No. 122799, PerkinElmer, Waltham, MA) was administered via intraperitoneal injection (IP; 15 mg/mL in DPBS −/− Ca²⁺Mg²⁺, 150 mg/kg of body weight). Luciferase transgene expression was imaged using the IVIS Lumina LT Series III pre-clinical in vivo imaging system (PerkinElmer, Waltham, MA) and whole body and tissue-specific luminescence quantitated using Living Image (PerkinElmer, Waltham, MA) software. Tissues from dosed CD1 mice were analyzed by eGFP immunohistochemistry.

### Animal Procedures for NHP.

Housing and all procedures were in accordance with the IACUC. All breeding, housing, and procedures were performed on cynomolgus macaques (Macaca fascicularis) as previously described (see, Ellsworth et al., PLoS One (2019) 14(11):e0225582). Sera from all animals were prescreened for anti-AAVHSC neutralizing antibodies using the Huh-7 cell-based assay (Horae Gene Therapy Center, University of Massachusetts, Worchester, MA) over a range of serum dilutions from 1/10 to 1/1250. Only antibody negative animals were used in this work. Each subject was anesthetized (young males, 4-5 months old) with an intramuscular injection (IM) of ketamine (10 mg/kg). Each animal was fitted with a saphenous vein catheter for IV injection of AAVHSCs. Blood samples were collected from the femoral vein for assessment of clinical chemistries, CBCs, and neutralizing antibodies at baseline. AAVHSCs were infused at 7-8 mL/kg through a saphenous vein catheter over 1.0 minute using all-plastic syringes. Each animal was allowed to recover in a warmed incubator following injection and, when fully awake was returned to the cage with its mother. At 2 weeks post-dosing, all subjects were anesthetized with ketamine, heparinized (5,000 units, given 5-10 minute prior to sacrifice) and euthanized with an overdose of IV sodium pentobarbital (80-100 mg/kg). ALT and AST levels in the serum of dosed NHPs were determined at VRL Laboratories (San Antonio, Texas) using a Clinical CBC (complete blood count) and Chemistry panel.

DNA Isolation from Murine Tissues.

DNA was extracted from tissues by the Maxwell RSC (Promega, Madison, WI) using the Maxwell RSC DNA FFPE Kit (Promega, Madison, WI) and by the QIAamp® Fast DNA Tissue Kit (Qiagen, Hilden, Germany) both as described by the manufacturer with the following modifications: Approximately 50 mg of tissue was homogenized in Precellys Soft tissue homogenizing CK14 2 mL tube with the Precellys Evolution homogenizer (Bertin Instruments, Montigny-le-Bretonneux, France) for 30 seconds at 5,000 rpm. DNA was eluted in 50 μL Ultrapure water.

DNA Isolation from Fixed NHP Tissue.

Approximately 30 g portions of fixed NHP tissue were isolated using a biopsy punch. Samples were homogenized in Precellys Soft tissue homogenizing CK14 2 mL tube with the Precellys Evolution homogenizer (Bertin Instruments, Montigny-le-Bretonneux, France) for 30 seconds at 5,000 rpm. Homogenized tissue was then digested for 24 hours using Proteinase K (ThermoFisher Scientific, Waltham, MA) and DNA was isolated using the RecoverAll™ Total Nucleic Acid Isolation Kit (ThermoFisher Scientific, Waltham, MA). DNA was eluted in 50 μL Ultrapure water (New England Biolabs, Ipswich, MA).

### Quantitation of VG Biodistribution in Animal Model Tissues.

VG copies per allele were determined by digital droplet PCR (ddPCR) using ddPCR Supermix for Probes (No dUTP) (Bio-Rad Laboratories, Hercules, CA). Detection of targets in 10 ng of sample input was accomplished using the primers and probes set forth in Table 3 (Integrated DNA Technologies, Coralville, IA):

TABLE 3
Primers and Probes for ddPCR

| | | | SEQ |
|---|---|---|---|
| | | | ID |
| | | Sequence | NO: |
| eGFP | Forward | 5′-CTGCTGCCCGACAACCA-3′ | 434 |
| #1 | Reverse | 5′-GACCATGTGATCGCGCTTCT-3′ | 435 |
| | Probe | 5′-TACCTGAGCACCCAGTCCGCCCT-3′ | 436 |
| eGFP | Forward | 5′-GAACCGCATCGAGCTGAA-3′ | 443 |
| #2 | Reverse | 5′-TGCTTGTCGGCCATGATATAG-3′ | 444 |
| | Probe | 5′-ATCGACTTCAAGGAGGACGGCAAC-3′ | 445 |
| Luci- | Forward | 5′-CTCAAAGTATTCAGCATAGGTGA | 446 |
| ferase | | TGTC-3′ | |
| | Reverse | 5′-AACTGCACAAGGCCATGAAGA-3′ | 447 |
| | Probe | 5′-TTGCCTTCACTGATGCTCACATTG | 448 |
| | | AGGT-3′ | |
| Mouse | Forward | 5′-CGTGGGCTCCAGCATTCTA-3′ | 449 |
| ApoB | Reverse | 5′-TCACCAGTCATTTCTGCCTTTG-3′ | 450 |
| | Probe | 5′-CCTTGAGCAGTGCCCGACCATTG-3′ | 451 |
| NHP | Forward | 5′-TGAAGGTGGAGGACATTCCTCTA-3′ | 437 |
| ApoB | Reverse | 5′-CTGGAATTGCGATTTCTGGTAA-3′ | 438 |
| | Probe | 5′-CGAGAATCACCCTGCCAGACTT | 452 |
| | | CCAT-3′ | |

Post droplet generation, 96-well plates were sealed with an adhesive plate film, transferred to a thermocycler for cycling, and then to the QX200 Droplet Reader for analysis. Data were analyzed using QuantSoft Analysis Pro Software (Bio-Rad Laboratories, Hercules, CA).

### Histology and Microscopy.

Paraffin-embedded liver tissues were sectioned at 4-5 μM and were processed for eGFP immunohistochemistry (IHC) as previously described (see, Ellsworth et al., PLoS One (2019) 14(11):e0225582). eGFP-positive cells were stained by IHC with a rabbit anti-eGFP polyclonal antibody (Item No. ab290, Abcam, Cambridge, United Kingdom). Endogenous peroxidase was inhibited by incubation in 3% H2O2. Samples were pretreated with proteinase K, followed by a serum-free protein block (epitope retrieval). Primary antibody incubation for 30 minutes at room temperature, followed by an EnVision+ Rabbit HRP detection system (Agilent, Santa Clara, CA). eGFP was visualized with an EnVision+ Rabbit HRP kit (Agilent, Santa Clara, CA) using diaminobenzidine to stain for eGFP and hematoxylin as counterstain. Slides were viewed with a Nikon Eclipse 80i microscope with an attached Nikon DXM 1200C digital camera (Nikon Instruments Inc., Melville, NY). IHC images were acquired using Aperio-Imagescope (Leica Biosystems, Wetzlar, Germany) and Huron Viewer (Huron Digital Pathology, Ontario, CA) software.

### Statistical Analysis.

All statistical analysis was performed using GraphPad Prism software using either a one-way or two-way analysis of variance (ANOVA) test.

### Example 1: Variations in AAVHSCs Lead to Different Glycan Affinity and Transduction Efficacy

AAVHSCs belong to Clade F, a clade that utilizes terminally exposed galactose for cellular binding. To determine if the naturally occurring variations on the AAVHSCs affect their binding, AAVHSC binding to CHO cell lines was examined. Lec2 are the mutant form of their parent, Pro5, and bear a mutation that reduces translocation of CMP-sialic acid, leading to high levels of surface exposed galactose on Lec2 (FIG. 2A). High and low levels of terminal galactose on Lec2 and Pro5 cells, respectively, were confirmed. Pro5 and Lec2 cells were transduced at a MOI of 2×10⁴ on ice for 1 hour. Binding was performed on ice to prevent bound AAV internalization and cells were washed with ice cold PBS to remove unbound particles prior to membrane bound vector genome (vg) analysis. For eGFP expression, cells were resuspended in media post washing, incubated overnight at 37° C. and assayed at 24 hours by flow cytometry.

All AAVHSCs displayed improved binding and transduction of Lec2 cells with terminally exposed galactose over the parent cell line Pro5, apart from AAVHSC16 (FIGS. 2B-2C). AAVHSC16 showed no difference in the number of vgs bound or eGFP expression for Lec2 and Pro5 cells, indicating AAVHSC16 does not share the galactose-binding feature of Clade F capsids. AAVHSC16 contains two unique residues compared to AAV9 and other AAVHSCs, 501I and 706C (Table 4). Both residues are surface exposed and therefore could affect either glycan and/or receptor binding.

TABLE 4
Amino Acid Variations in AAVHSCs with Respect to AAV9

| | Capsid Region | | |
|---|---|---|---|
| AAVHSC Serotype | VP1 | VP2 | VP3 |
| AAVHSC1 | A2T | — | — |
| AAVHSC3 | — | G161D | — |
| AAVHSC4 | F119L | — | P468S |
| AAVHSC6 | — | — | Q590R |
| AAVHSC7 | A68V | — | — |
| AAVHSC8 | — | Q151R | — |
| AAVHSC9 | — | — | C206G |
| AAVHSC13 | — | — | G505R |
| AAVHSC15 | — | — | G505R, T346A |
| AAVHSC16 | — | — | F501I, G505R, Y706C |
| AAVHSC17 | — | — | G505R |

While all AAVHSCs except for AAVHSC16 preferentially bound the Lec2 cells with terminal galactose exposed, the fold change in binding was different between two groups of AAVHSCs. It was observed that the AAVHSCs containing the 505R residue (AAVHSC13, 15, 16, and 17) had a lower fold binding enhancement in Lec2 than the capsids with 505G (AAVHSC1, 3, 4, 6, 7, 8, 9) (Table 4 and FIG. 2D). These data indicate that galactose binding was stronger in capsids with a glycine at residue 505 and that arginine negatively impacted galactose binding. The 505 residue is modeled within a loop on the surface of the capsid and part of a protrusion surrounding the galactose-binding pocket. The glycine to arginine variation increases the size and adds a positive charge at 505 and may affect galactose-binding due to either steric hindrance or difference in charge. AAVHSC4 displayed an increase in binding to galactose but had little eGFP expression in both cell lines (FIGS. 2B-2C).

### Example 2: Isoleucine at 501 Residue on AAVHSC16 is Responsible for Altered Terminal Galactose Binding

The contribution of each unique residue on AAVHSC16, 501I or 706C, in lowered galactose binding was investigated by mutagenizing the 501 and 706 residues on AAVHSC15, an AAVHSC that also contains 505R (see, Table 4 and 5) and the most studied capsid in the AAVHSC panel. As AAVHSC16 did not have robust binding or transduction of Pro5 or Lec2 cells, additional mutant CHO cell lines were included to assess AAVHSC16 and mutagenized capsid binding to other terminal glycans (FIG. 2A). The lack of terminal galactose was confirmed in Lec1 and Lec8 cells. Pro5, Lec1, Lec2, and Lec8 cells were transduced at a MOI of 5×10³ on ice for 1 hour. Cells were washed 3 times post transduction with cold PBS to determine surface bound vector genomes. GFP expression was determined 24 hours post incubation at 37° C. post washing.

TABLE 5
Amino Acid Mutagenesis of AAVHSC Capsid Proteins

| AAVHSC Serotype | Amino Acid Variation |
|---|---|
| AAVHSC7 | F501I |
| AAVHSC15 | F501I |
| AAVHSC15 | Y706C |
| AAVHSC16 | I501F |
| AAVHSC16 | C706Y |
| AAVHSC16 | I501L |
| AAVHSC16 | I501T |
| AAVHSC16 | I501V |
| AAVHSC16 | I501W |
| AAVHSC16 | I501Ya or “c1” |
| | (codon TAT) |
| AAVHSC16 | I501Yb or “c2” |
| | (codon TAC) |
| AAVHSC16 | T346A |

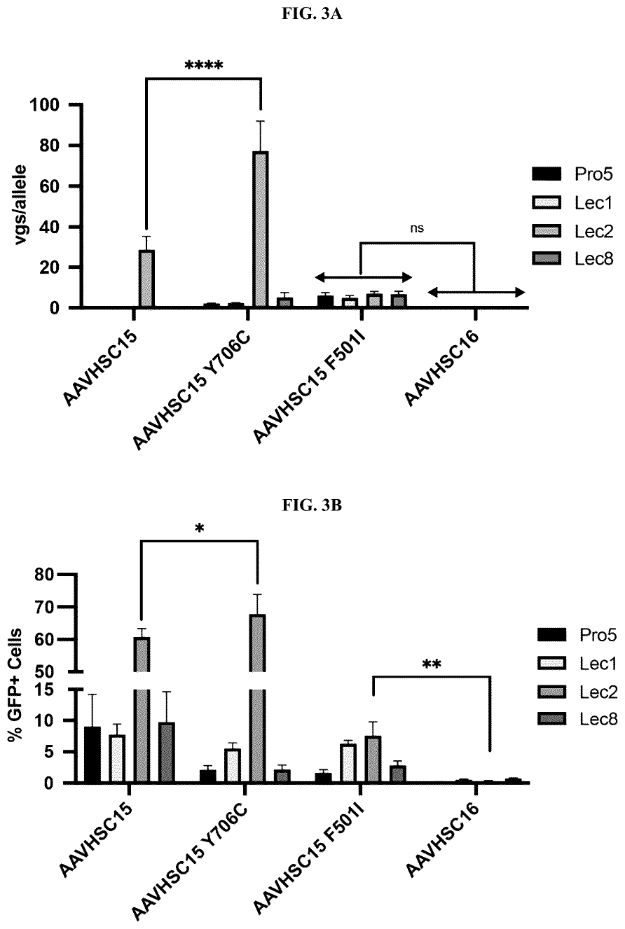

AAVHSC15 and AAVHSC16 did not display an increase in binding to Lec1 or Lec8 cells over Pro5 cells, indicating a lack of preferential binding to terminal GlcNAc (N-acetylglucosamine) or mannose (FIG. 3A). The binding pattern of AAVHSC15 with the Y706C variation was similar to AAVHSC15, with only increased binding to galactose observed. AAVHSC15 with the F501I variation mimicked the binding pattern of AAVHSC16, with no preferential binding to any of the terminal glycans on the CHO cell lines. These data indicate that the 501I residue on AAVHSC16 is the main contributor to the abolished galactose binding and supports previously published work on key residues of Clade F AAVs that influence galactose binding. Although 501, 505, and 706 residues are located on the surface of the capsid, only 501 and 505 are in close proximity to key galactose-binding residues of AAV9. While the pattern between AAVHSC15 and AAVHSC15 Y706C was similar, AAVHSC15 Y706C had significantly increased galactose binding and expression in Lec2s compared to wild-type (wt) AAVHSC15, indicating that a variation not in proximity to the galactose binding pocket altered binding and transduction of cells with high levels of surface exposed galactose (FIGS. 3A-3B).

AAVHSC15 F501I expressed in all CHO cell lines better than AAVHSC16 (FIG. 3B), indicating that remaining variations between these capsids, the 346 and 706 residues (Table 4), contribute to additional functional differences. These variations led to detectable eGFP expression (1 to >10%) with AAVHSC15 and 501 and 706 variants in cell lines with low levels of bound vgs. The highly efficient eGFP expression of AAVHSC15 and AAVHSC15 501 and 706 variants may be due to the T346A residue, which is not found on AAVHSC16. The 346 residue is located internally on the β-strand of the capsid and may play a role in intramolecular interactions, altering expression. 346T is highly conserved among serotypes including AAV2, AAV3b, AAV5, AAV6, AAV8, and AAV9.

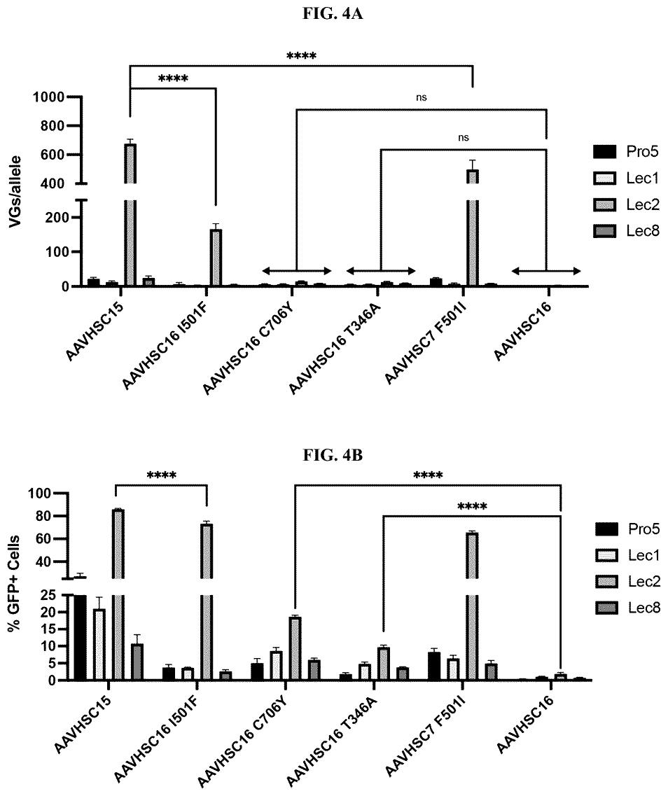

To further confirm the contribution of 501I on AAVHSC16 to the lack of galactose binding, we generated and tested gain of function mutants AAVHSC16 I501F and AAVHSC16 C706Y for their binding and transduction efficiency on CHO cell lines (FIGS. 4A-4B). In addition to the AAVHSC16 gain of function mutants, the influence on glycan binding of the 501I residue was investigated on a non-505R AAVHSC (AAVHSC7, Table 4 and 5) as well the effect of the unique AAVHSC15 346A residue on transgene expression of AAVHSC16. AAVHSC16 Y706C did not display increased binding to surface exposed galactose but did result in significantly increased expression in Lec2s, indicating a post-attachment role of the Y706C variation for AAVHSC16. While the AAVHSC16 I501F displayed partially restored binding to terminal galactose and expression in Lec2 cells, the number of bound vgs and GFP expression were still significantly lower than wt AAVHSC15. This difference in binding and GFP expression between wt AAVHSC15 and AAVHSC16 I501F once again highlights the contribution of the unique 346 and 706 residues to functional differences between the capsids. The contribution of 346A, which is located internally on the capsid, to AAVHSC15 expression was highlighted in the AAVHSC16 T346A variant, where AAVHSC16 T346A showed higher GFP expression over wt AAVHSC16 with no significant increase in membrane bound vgs. Interestingly, AAVHSC7 F501I retained the ability to bind terminal galactose and transduce Lec2 (FIGS. 2B-2C and 4A-4B).

### Example 3: Amino Acid at 501 Residue on AAVHSC16 Contributes to Overall Binding Function

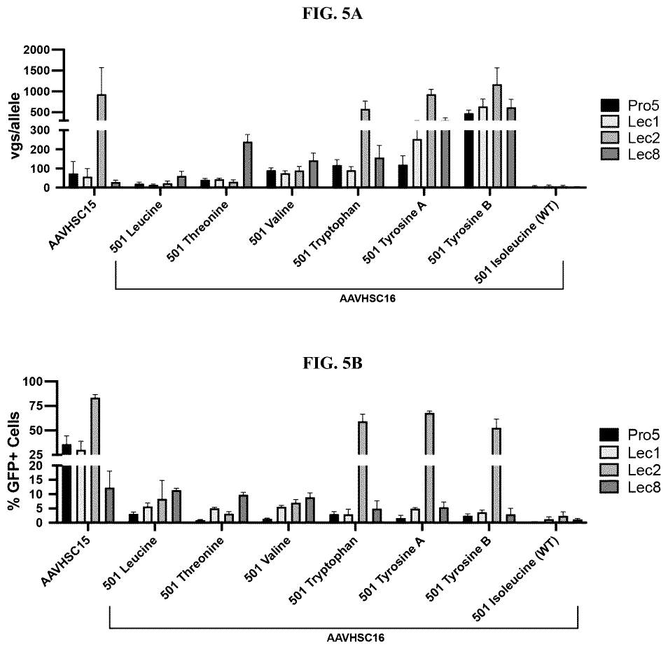

To test the specific contribution of isoleucine at the 501 residue to galactose binding, 501I on AAVHSC16 was mutagenized to leucine, threonine, valine, tryptophan, and two different tyrosine codons (Table 5). Pro5, Lec1, Lec2, and Lec8 cells were transduced at a MOI of 2×10⁴ (for FIGS. 5A-5B) on ice for 1 hour. Cells were washed 3 times post transduction with cold PBS to determine surface bound vector genomes (FIG. 5A). GFP expression was determined 24 hours post incubation at 37° C. post washing (FIG. 5B).

Leucine and valine are also hydrophobic amino acids with leucine similar in size to isoleucine, but both larger than valine due to an extra carboxyl group. Leucine is as hydrophobic as phenylalanine, and both are more hydrophobic than valine and isoleucine. Threonine and valine have a similar branch structure with the exception of a hydroxyl side chain but threonine and isoleucine are the only amino acids with two stereogenic centers. Tryptophan and tyrosine both contain an aromatic ring structure in their side chain, making them most similar in structure to phenylalanine, with tryptophan being a binuclear ring structure and the largest of all amino acids. Modeling of the mutagenized 501 residues on AAVHSC16 revealed that aromatic ring side chain of tryptophan and tyrosine side is predicted to occupy similar space in the pocket to phenylalanine. Binding analysis of 501 mutagenized AAVHSC16 capsids revealed that leucine, threonine, and valine variants have preferential binding to terminal GlcNAc of Lec8 cells, indicating a shift in glycan binding with leucine, threonine, or valine at 501 (FIG. 5A). This increase in binding to the Lec8 cells also led to increased eGFP expression for the leucine, threonine, or valine AAVHSC16 mutants. Interestingly, data previously published showed wheat germ agglutinin (WGA), which binds to GlcNAc, slightly reduced other Clade F AAV binding to Pro5 cells, indicating the leucine, threonine, or valine at the 501 position may be enhancing Clade F GlcNAc binding. The introduction of an aromatic ring at the 501 position with tryptophan or tyrosine allowed the AAVHSC16 I501W and I501Y variants to bind all terminal glycans along with restoration of preferential binding to terminal galactose as observed with other AAVHSCs containing a phenylalanine at 501.

eGFP expression was elevated in all CHO cells lines with 501 mutagenized AAVHSC16s compared to AAVHSC16, correlating with the improvement in overall binding (FIG. 5B). Although tryptophan and tyrosine were able to bind to all the terminal glycans of all the CHO cell lines at high efficiencies, only the Lec2 cells displayed high GFP expression at levels close to AAVHSC15, which also contains an aromatic ring at 501 with phenylalanine. No significant differences in binding or GFP expression was observed between AAVHSC16 YA and YB, indicating that the codon usage and tRNA availability did not alter this specific function. Differences observed here in binding versus expression are aligned with published data highlighting the 501 on AAV9 as a key residue for galactose binding and post-attachment processes up to expression. These data also imply that the specific amino acid and side chain at 501 influences the function of the virus.

### Example 4: AAVHSC16 has Significantly Reduced Hepatic Transduction in In Vivo and In Vitro Models

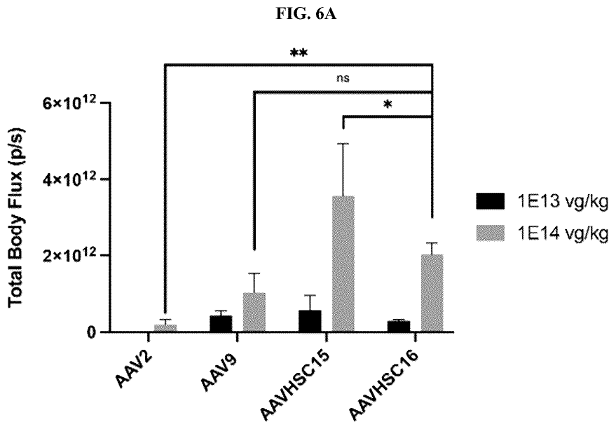

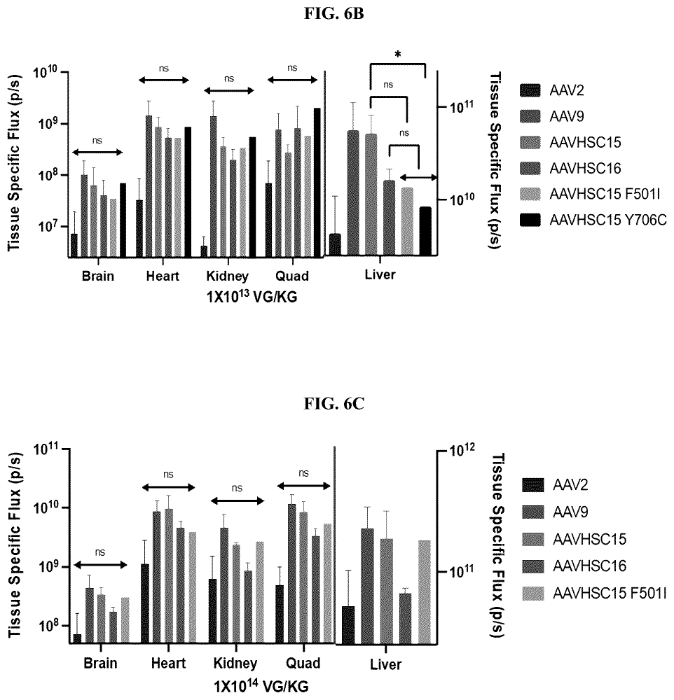

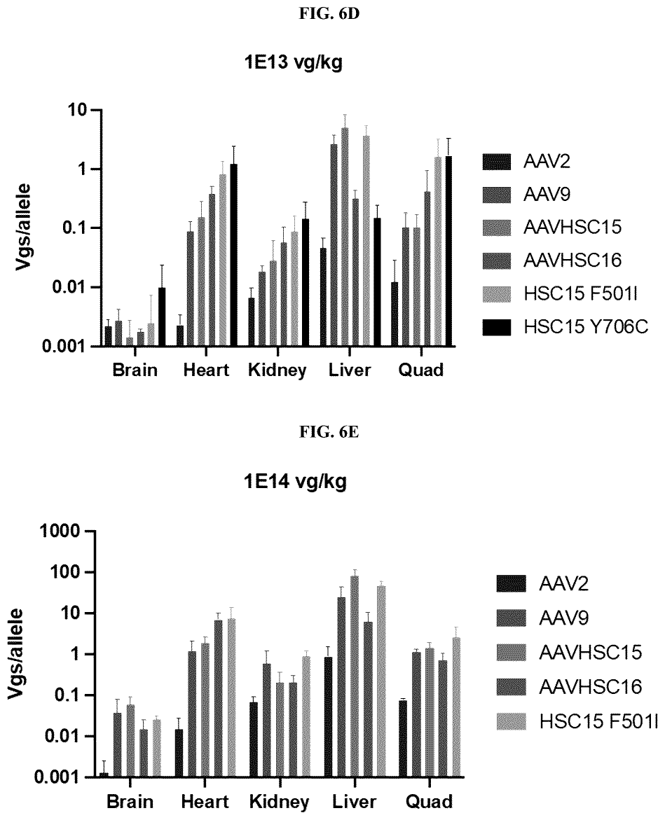

Adachi et al. observed that modulations to the 501 residue of AAV9 not only influenced glycan binding but also the in vivo tropism of that capsid. To test if differences in galactose-binding observed in vitro affect tropism in vivo, AAVHSC15 and AAVHSC16 were compared with two serotypes with weak and strong galactose binding, AAV2 and AAV9, respectively. Systemic biodistribution was assessed in albino C57 mice administered 1×10¹³ or 1×10¹⁴ vg/kg ssCBA.Luc vector I.V. At six weeks post dosing, total body bioluminescence was determined through imaging (FIG. 6A).

Total body bioluminescence revealed that AAV2, which does not bind galactose, had the lowest level of systemic expression at all doses tested (FIG. 6A). On the contrary, AAVHSC16 had comparable levels of total body expression to AAV9, despite their differences in galactose binding. This maintenance of total body expression for AAVHSC16 suggests that galactose binding is not sufficient to predict the overall systemic tropism in vivo. AAVHSC15, which binds strongly to terminal galactose, had the highest level of total body bioluminescence for both doses. To further characterize the tissue-specific tropism of AAV2, AAV9, AAVHSC15, AAVHSC16, AAVHSC15 F501I, and AAVHSC15 Y706C, key therapeutically relevant organs were assessed for bioluminescence post-harvest as well as cellular vgs (FIGS. 6B-6E). The liver was the only tissue with significant differences in bioluminescence observed between the capsids tested. AAVHSC16 lead to significantly lower levels of transgene expression in the liver compared to both AAV9 and AAVHSC15, with hepatic bioluminescence levels being insignificant to AAV2 at all doses tested. This lower liver tropism of AAVHSC16 ex vivo was in contrast to the whole body luminescence images, which displayed higher levels of bioluminescence located in the abdomen. It is possible that other tissues or fluids in the abdomen are contributing to this localized higher transgene expression for AAVHSC16. By examining the ratio of vgs among the different organs assayed, it became evident that AAVHSC16 also had a different biodistribution pattern with a lower ratio of vgs in the liver compared to the other Clade F AAVs tested. The ratio of vgs for AAV2 also revealed that it was less hepatic driven than galactose-binding AAV9 and AAVHSC15 with the lowest number of hepatic vgs. While AAVHSC16 had higher hepatic vgs than AAV2, approximately 6-fold higher for both doses, AAVHSC16 had between 4- and 16-fold lower liver vgs compared to AAV9 and AAVHSC15. In contrast to the significantly lower liver tropism, AAVHSC16 maintained high levels of transduction in the CNS and periphery, with equivalent vgs as AAV9 and AAVHSC15 in these tissues (FIGS. 6D-6E).

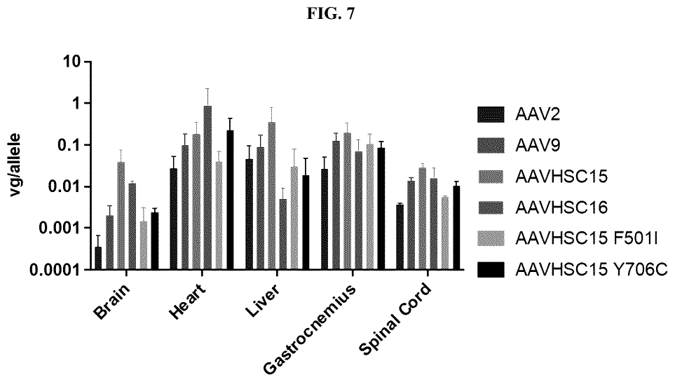

To further characterize tissue-specific expression of AAVHSC16 compared to AAV2, AAV9, AAVHSC15, AAVHSC15 F501I, and/or AAVHSC15 Y706C, CD1 mice were injected I.V. with 7.5×10¹² vg/kg of scCBA.eGFP vector with eGFP expression and vgs determined at 4 weeks post dose (FIG. 7). A different strain and dose were chosen to determine if the lower hepatic transduction was influenced by either variable. Liver vgs were found to be lower with AAVHSC16 compared to AAV9, AAVHSC15, and unexpectedly, AAV2. Hepatic GFP expression among the different capsids correlated with hepatic vgs (FIG. 7). AAVHSC16 vgs were between 2- and 3-fold lower in the brain, spinal cord, and gastrocnemius compared to AAVHSC15 yet were comparable or slightly higher than AAV9 in these tissues. AAV9 displayed the most robust GFP expression in the peripheral tissues (heart, liver, gastrocnemius) but lead to drastically lower GFP expression in the CNS tissue (brain and spinal cord) compared to AAVHSC15 and AAVHSC16. Although AAVHSC16 had higher vgs in the heart compared to AAVHSC15 and AAV9, expression appeared lower with AAVHSC16. Differences in sampling of the heart for vg and expression analysis or capsid specific expression mechanisms of action may explain this discrepancy. The vg and eGFP expression analysis of AAVHSC16 demonstrates the maintenance of broad biodistribution of this AAVHSC with lowered liver tropism.

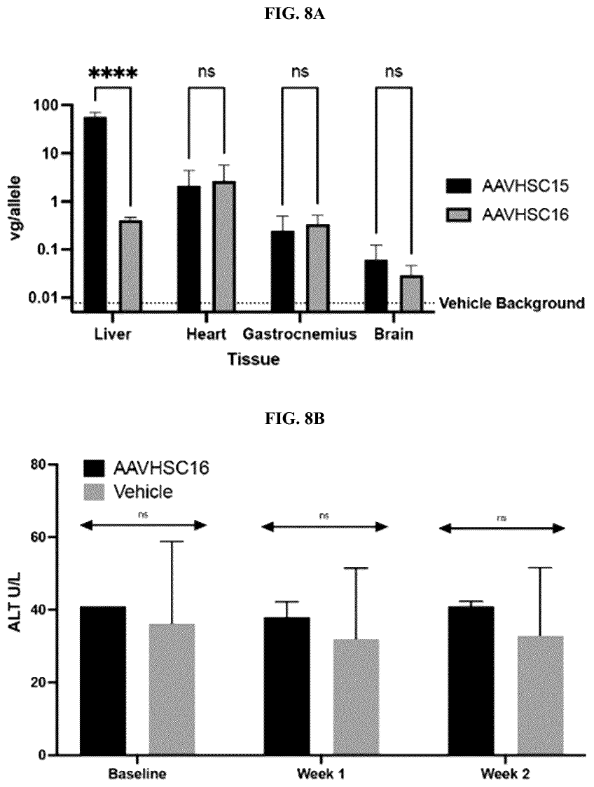

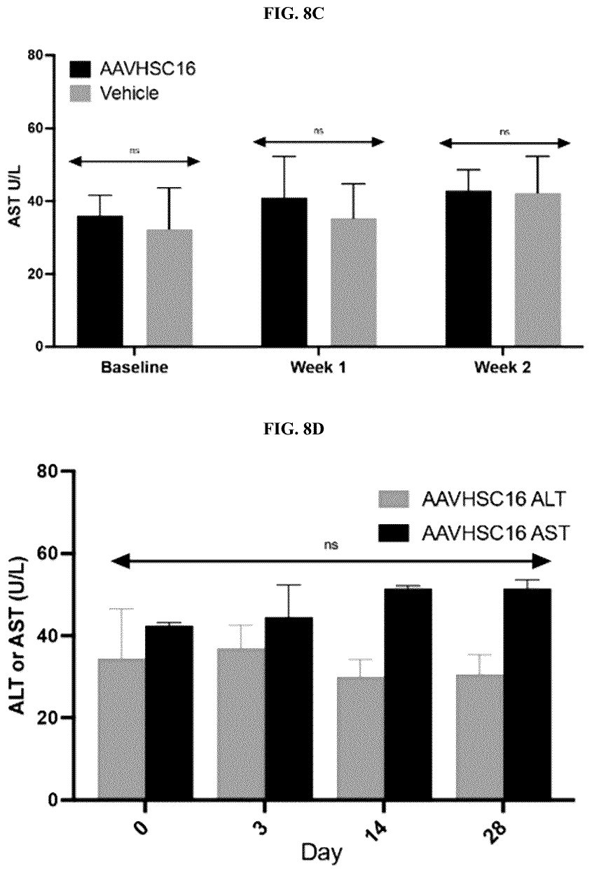

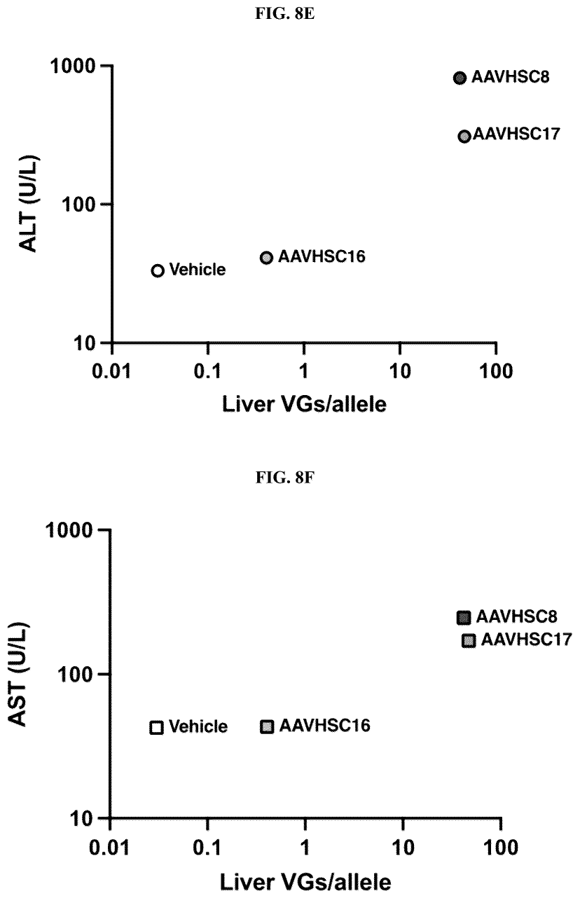

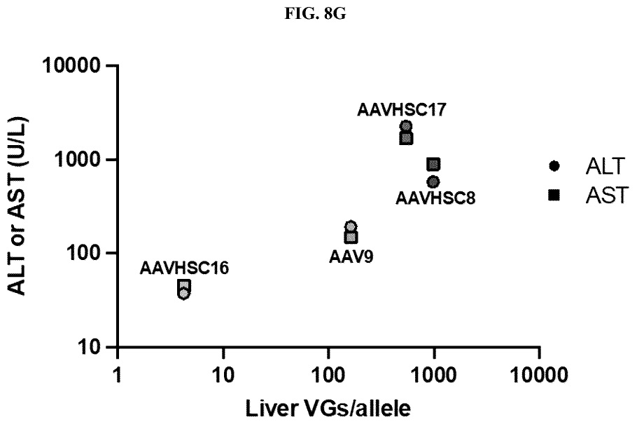

To determine if the unique tropism of AAVHSC16 is species specific, we investigated biodistribution in NHPs. Biodistribution of AAVHSC15 and AAVHSC16 was examined in two cynomolgus macaques (Macaca fascicularis) per capsid at 2 weeks post I.V. delivery of scCBA.eGFP vector at a 7×10¹³ vg/kg dose. eGFP expression was substantially greater in the liver of NHPs with AAVHSC15 compared with those that received AAVHSC16. NHPs administered AAVHSC16 had approximately three logs fewer hepatic vgs than NHPs administered AAVHSC15 (FIG. 8A). Despite variability between individual NHPs, AAVHSC16 maintained high and equivalent levels of transduction (vgs) and expression in the heart, gastrocnemius and brain compared to AAVHSC15, indicating high levels of non-hepatic and broad systemic tropism in vivo were not species restrained. The reduced hepatic tropism of AAVHSC16 also did not lead to increased levels of liver transaminases above baseline or compared to vehicle controls (FIGS. 8B-8D). In addition, NHPs administered AAVHSC16 scCBA.eGFP at 7×10¹³ vg/kg or a vector expressing human phenylalanine hydroxylase driven by a liver specific promoter (ssDNG.PAH) at 1×10¹⁴ vg/kg did not display elevated ALT or AST levels at any timepoint post dose compared to baseline levels (FIG. 8D). Supporting the finding that the reduced liver tropism of AAVHSC16 led to reduced liver toxicity, it was found that liver-tropic capsids AAV9, AAVHSC8, and AAVHSC17 led to increased ALT and AST levels in NHPs (FIGS. 8E-8G).

In an effort to translate the lowered liver tropism of AAVHSC16 observed in the mouse and NHPs to human cells, primary hepatocytes were transduced with several wt AAVHSC and AAVHSC variant scCBA.eGFP vectors at three multiplicities of infection (MOIs). At four days post transduction, AAVHSC15, AAV9, AAVHSC7, and AAVHSC7 F501I led to greater eGFP expression than AAVHSC16 and all AAVHSC16 variants at all MOIs tested. As observed in vitro with the CHO cells, the internally located 346A residue change alone on AAVHSC16 increased expression in primary human hepatocytes. The AAVHSC15 variants containing either the AAVHSC16 501I or 706C revealed that the lower hepatocyte expression from AAVHSC16 is primarily due to the 706C variation and not the galactose altering 501I variation. The AAVHSC16 gain of function mutants also revealed that the variation at the 706 residue was the main contributor to lowered hepatocyte expression, as AAVHSC16 C706Y had more expression restored than AAVHSC16 I501F. While all 501 changes (L, T, V, W, and Y) partially restored expression of AAVHSC16 in the hepatocytes, the largest and most intrusive 501 change with tryptophan restored the least amount of hepatic eGFP expression. The lack of eGFP expression observed with AAVHSC16 in primary human hepatocytes along with the AAVHSC16 501 mutants reinforced that the significantly reduced hepatic tropism can be translated among species and that all unique amino acid variations contribute to the unique binding and biodistribution of AAVHSC16.

### Example 5: Naturally Occurring Variations at the 501 and 706 Residues on AAVHSC16 Contribute to Slower Serum Clearance

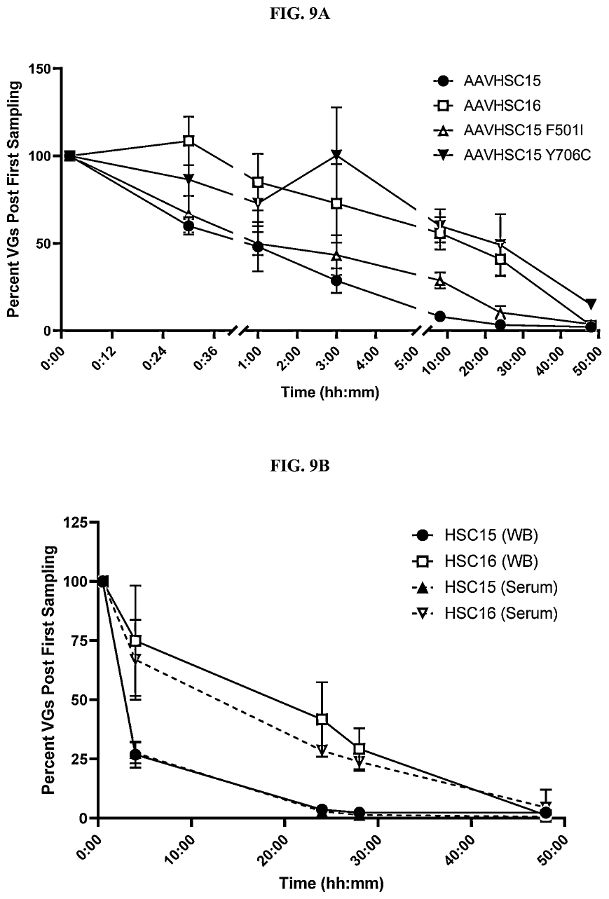

Blood and serum clearance of AAV was measured by quantifying AAV vectors by qPCR in serial blood draw samples taken at 2 minutes, 3 minutes, 1 hour, 3-4 hours, 8 hours, 24-28 hours, and 48 hours post-dose of B6 Albino mouse administered AAVHSC15, AAVHSC16, AAVHSC15-F501I, or AAVHSC15-Y706C scCBA-GFP vectors via retro-orbital injection at 7.5×10¹² vg/kg. As shown in FIG. 9A, mice administered AAVHSC16 had slower clearance of vector genomes from the blood compared to mice administered AAVHSC15. When the naturally occurring variations F501I and Y706C were mutated in AAVHSC15, mice administered AAVHSC15-F501I and AAVHSC15-Y706C resulted in slower clearance of vector genomes from the blood compared to mice administered AAVHSC15. As shown in FIG. 9B, mice administered AAVHSC16 had slower clearance of vector genomes from both whole blood and serum as compared to mice administered AAVHSC15.

### Example 6: Variation at Residue 505 on AAVHSC7 and AAVHSC8

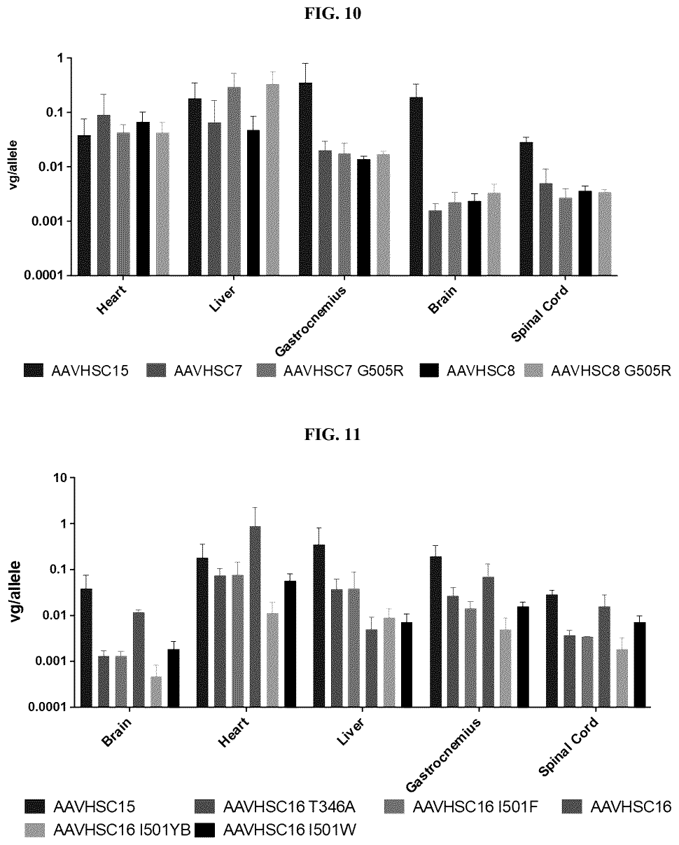

Based on the findings presented in Example 2, because AAVHSC7 has a glycine at residue 505, as opposed to the 505 arginine on AAVHSC15 and AAVHSC16, the findings indicate that residue 501I on AAVHSC16 contributes to the obliteration of terminal galactose binding, but only in combination with the 505R neighboring residue. To investigate the effect of 505R on non-505R AAVHSC, 505R was incorporated into AAVHSC7 and AAVHSC8. scCBA.eGFP vectors packaged in AAVHSC15, AAVHSC7, AAVHSC7-G505R, AAVHSC8, and AAVHSC8-G505R were intravenously administered into CD1 mice at a dose of 7.5e12 vg/kg. Tissue-specific transduction efficiency was determined by ddPCR 4 weeks post-administration (FIG. 10). As shown in FIG. 10, the incorporation of G505R to AAVHSC7 and AAVHSC8 elevated their ability to transduce liver cells without affecting their ability to transduce cells of the heart, muscle, brain, or spinal cord.

### Example 7: Variations at Residues 501 and 346 on AAVHSC16

As shown in Example 4, incorporating the internally located 346A to AAVHSC16 led to increased expression in primary human hepatocytes. Example 4 also demonstrated that the lower hepatocyte expression from AAVHSC16 is primarily due to the 706C variation and not the galactose altering 501I variation. To further investigate the function of residues 346 and 501, 346A and different 501 variations were incorporated into AAVHSC16. Residue 501 on AAVHSC16 was changed to tyrosine and tryptophan to reintroduce an aromatic ring (residue 501 is a phenylalanine on other AAVHSC capsids). scCBA.eGFP vectors packaged in AAVHSC15, AAVHSC16, AAVHSC16-T346A, AAVHSC16-I501F, AAVHSC16-I501YB (codon TAC), and AAVHSC16-1501W were intravenously administered into CD1 mice at a dose of 7.5e12 vg/kg. Tissue-specific transduction efficiency was determined by ddPCR 4 weeks post-administration (FIG. 11). As shown in FIG. 11, T346A and I501F on AAVHSC16 resulted in increased liver transduction and a decrease in the transduction of other tissues. When I501YB and I501W were incorporated into AAVHSC16, the lower liver targeting was maintained, but resulted in changes to the tropism of other tissues. AAVHSC16-1501W demonstrated higher transduction of the brain, heart, muscle, and spinal cord, compared to AAVHSC16-I501Y.

### Example 8: Transduction of Heart and Liver Cells

The ability of each of the capsids in Table 5, as well as AAV9, AAVHSC1, AAVHSC3, AAVHSC4, AAVHSC6, AAVHSC7, AAVHSC8, AAVHSC9, AAVHSC13, AAVHSC15, AAVHSC16, and AAVHSC17 capsids, to transduce heart and liver cells was investigated. Rat H9C2 cardiomyoblasts, Human Ac16 cardiomyocytes, and primary human hepatocytes were transduced with scCBA.eGFP vectors packaged in the various capsids under the conditions set forth in Table 6.

TABLE 6
Transduction Conditions

| | Rat | Human | |
|---|---|---|---|
| | Cardiomyo- | Cardiomyo- | Primary |
| | blasts | cytes | Human |
| Cell type | (H9C2) | (Ac16) | Hepatocytes |
| Cell density at seeding | 50k | 50k | 100k |
| Day transduced | Day 4 | Day 4 | Day 1 |
| Cell density at trans- | 75k | 300k | Same day |
| duction | | | |
| MOI | 6e5 | 6e5 | 1.5E5, 3E5 and 6e5 |
| Days at harvest | Day 6 | Day 6 | Day 5 |
| | | | (media change at |
| | | | Day 3) |

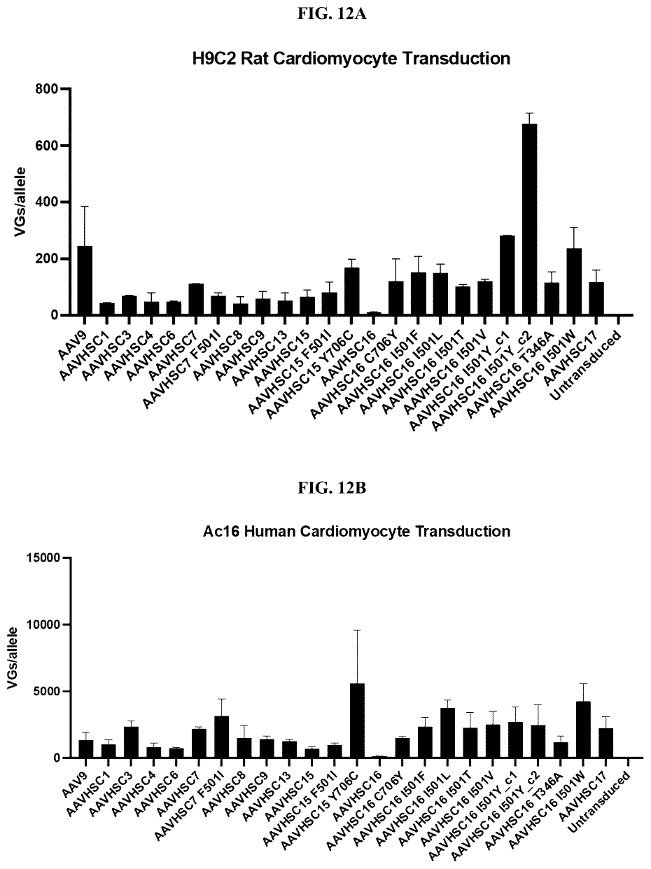

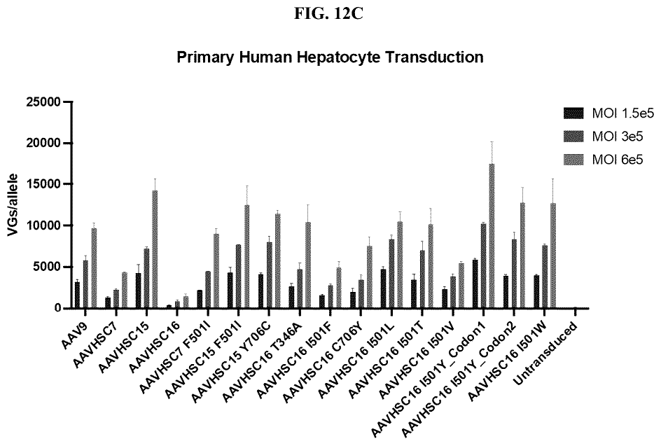

After harvest, vector genome analysis was performed as previously described. For H9C2 cells, vgs were determined by qPCR using primers and probes specific for GFP and rat GAPDH. For Ac16 cells and primary human hepatocytes, vgs were determined by qPCR using primers and probes specific for GFP and human ApoB. FIGS. 12A, 12B, and 12C show the vgs/allele of H9C2, Ac16, and primary human hepatocytes transduced with the various vectors, respectively.

A subset of the capsids tested in primary rat and human cells were investigated in vivo. scCBA.eGFP vectors packaged in the capsids set forth in Table 7 were intravenously administered into CD1 mice at a dose of 7.5e12 vg/kg.

TABLE 7
Capsids and Amino Acid Variations

| Capsid | Amino Acid Variation |
|---|---|
| AAV2 | None |
| AAV9 | None |
| AAVHSC7 | None |
| AAVHSC7 | G505R |
| AAVHSC8 | None |
| AAVHSC8 | G505R |
| AAVHSC15 | None |
| AAVHSC15 | F501I |
| AAVHSC15 | Y706C |
| AAVHSC16 | None |
| AAVHSC16 | T346A |
| AAVHSC16 | I501F |
| AAVHSC16 | I501Y |
| AAVHSC16 | I501W |

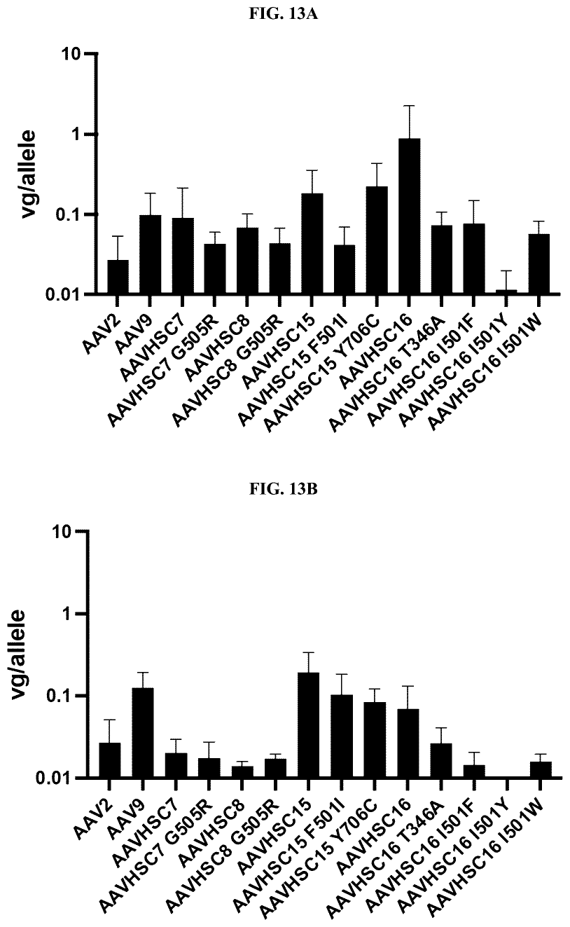

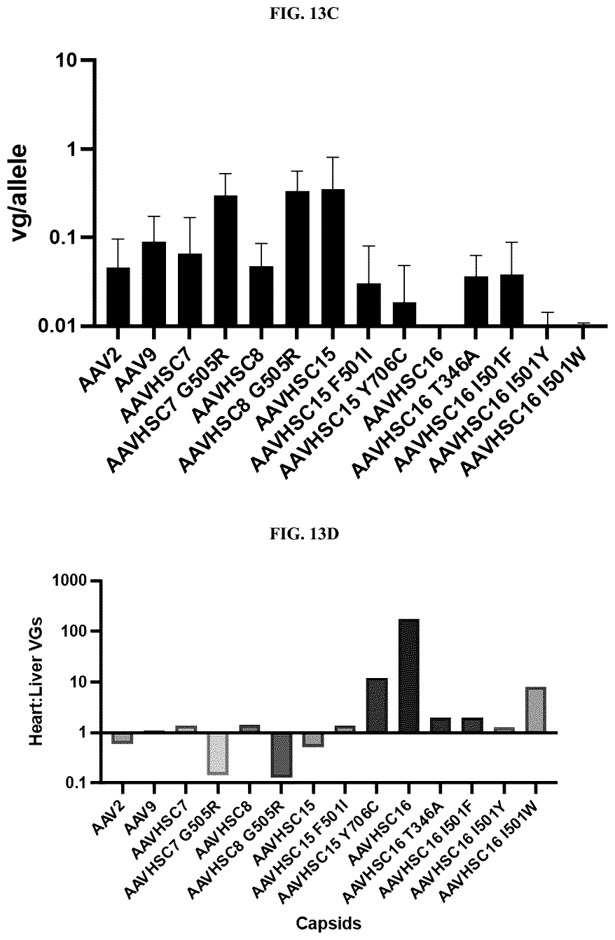

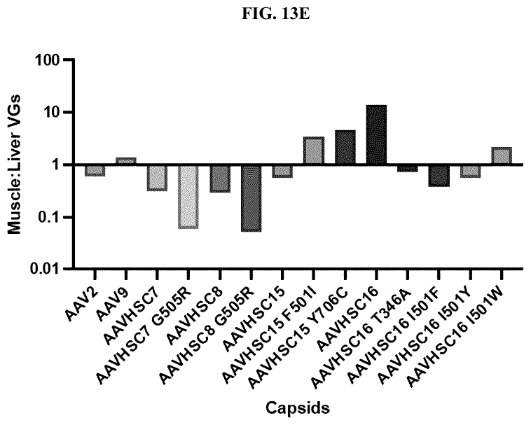

FIGS. 13A, 13B, and 13C show the transduction efficiency of heart, gastrocnemius, and liver, respectively, as determined by ddPCR 4 weeks post-administration. FIG. 13D shows the ratio of transduction efficiency of the various rAAV of heart to the transduction efficiency of the various rAAV to liver, and FIG. 13E shows the ratio of transduction efficiency of the various rAAV of muscle to the transduction efficiency of the various rAAV to liver.

### Example 9: Transduction of Various Tissues and Cross-Correction Potential by AAVHSC16

Lysosomal storage disorders (LSDs) are a group of genetic diseases that result in metabolic issues of the lysosome. LSDs typically are caused by the genetic loss of a catabolic enzyme, thereby causing the accumulation of the enzyme's substrate within the lysosome. Over time, tissue-specific accumulation of the substrate results in a spectrum of symptoms and disabilities that vary by LSD. LSDs are attractive targets for gene therapy because of the potential for cross-correction, a phenomenon that allows specific extracellular LSD enzymes to be taken up and targeted to the lysosomes of otherwise enzyme-deficient cells. The LSD mucopolysaccharidosis II (Hunter syndrome) is caused by a deficiency in iduronate-2-sulfatase (I2S) that is encoded by the IDS gene.

The ability of AAVHSC16 to transduce various tissues was investigated. Mice were intravenously administered a rAAV comprising a ubiquitous promoter operably linked to a transgene encoding IDS (single-stranded CMV-IDS) packaged in AAVHSC16 (“AAVHSC16-IDS”) at a dose of 1E12 vg/kg, 5E12 vg/kg, 1E13 vg/kg, 5E13 vg/kg, or 1E14 vg/kg. Animals were handled and tissues were processed as previously described. Vector genome copies per allele were determined by ddPCR with probes specific for the IDS transgene.

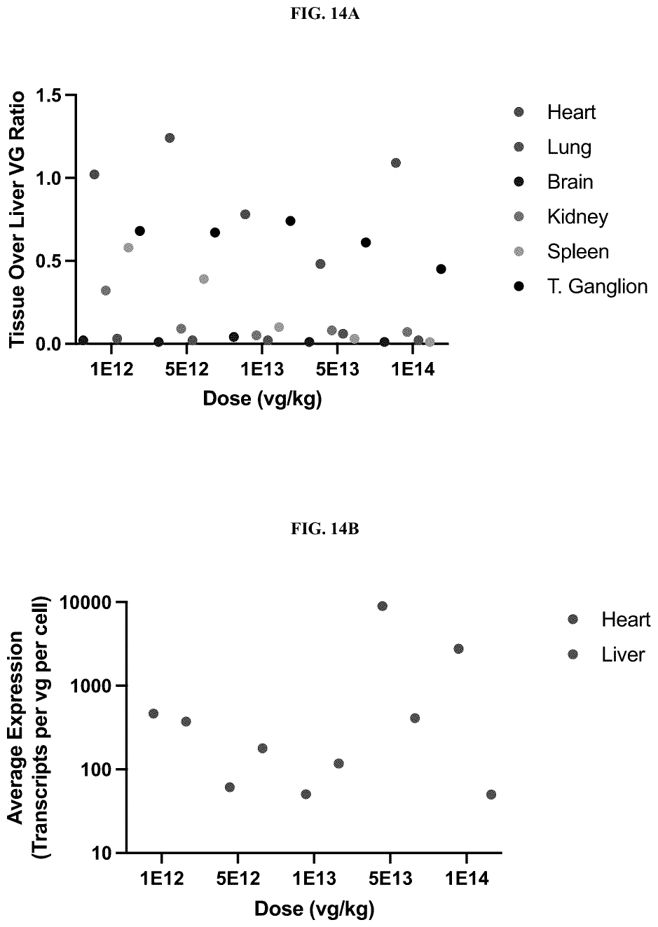

FIG. 14A is a graph showing the efficiency of AAVHSC16 to transduce heart, lung, brain, kidney, spleen tissues, as well as the trigeminal ganglion in the peripheral nervous system, presented as a ratio between the level of transduction of the various tissues to the level of transduction of liver tissue. Ratios were calculated by dividing the average vgs/allele of each specified tissue by the average vgs/allele in the liver. As shown in FIG. 14A, AAVHSC16 demonstrated a favorable heart:liver ratio with at least 0.5 vgs to every liver vg for all doses tested.

As shown in FIG. 14B, AAVHSC16 was capable of mediating a higher expression level (transcripts per vg per cell) in the heart than liver at higher doses (FIG. 14B).

In order to investigate the potential of AAVHSC16 to mediate cross-correction, mice were intravenously administered AAVHSC16-IDS at a dose of 1E12 vg/kg, 5E12 vg/kg, 1E13 vg/kg, 5E13 vg/kg, or 1E14 vg/kg. Animals were handled and tissues were processed as described in Example 1. Vector genome copies per allele were determined by qPCR with probes specific for the IDS transgene.

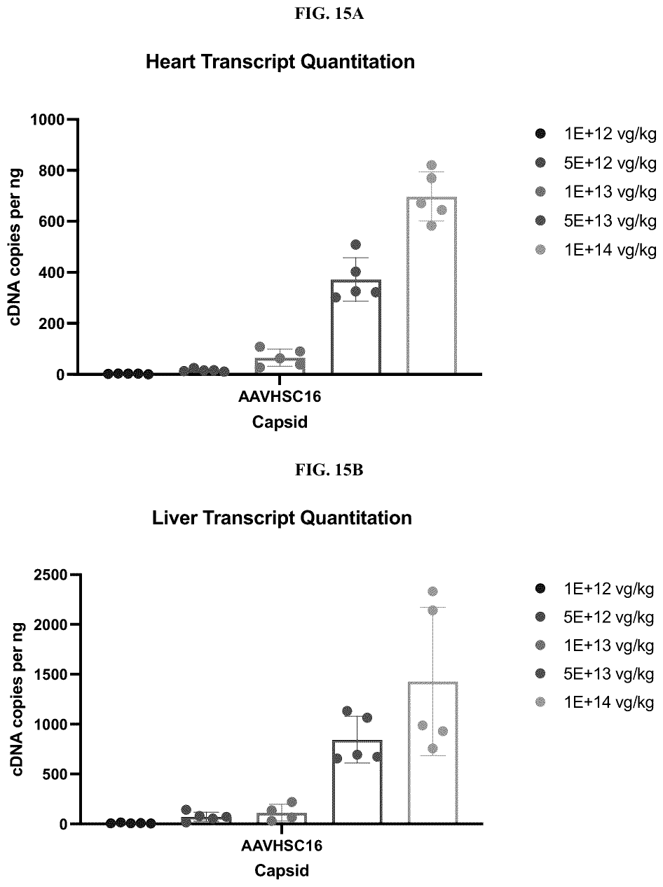

As shown in FIGS. 15A and 15B, AAVHSC16-mediated delivery of IDS resulted in dose-dependent IDS transcript expression in the heart (FIG. 15A) and liver (FIG. 15B) with no plateau observed at the doses tested.

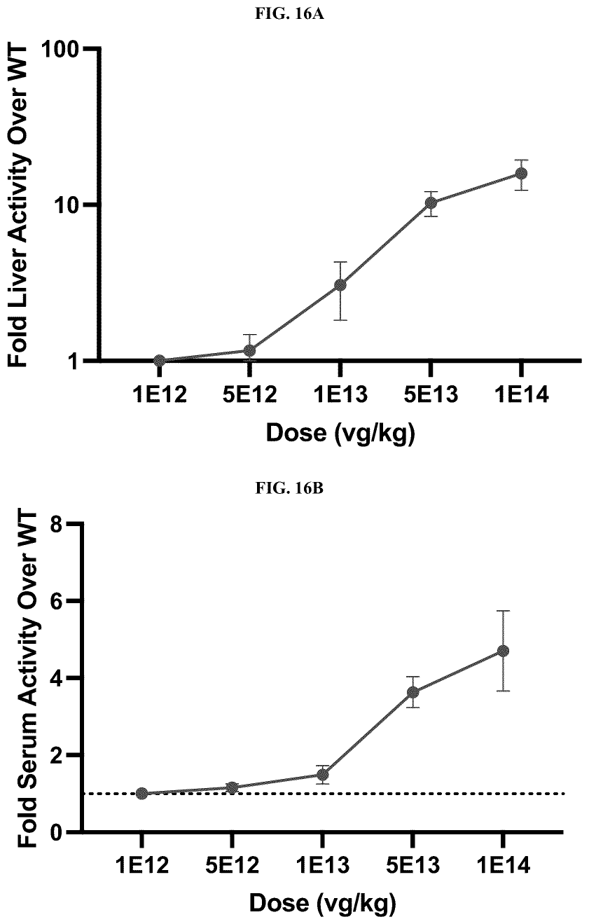

I2S activity in the liver and serum were determined using a two-step fluorometric assay. Briefly, tissues or serum were incubated with a fluorescently labeled α-L-iduronide-2-sulphate (4-MUS). After 4 hours, IDUA was added to the reaction which led to the release of the fluorescent final product 4-methylumbelliferone (4-MU). I2S activity was determined by a plate reader after reaction was excited at 365 nM and signal was read at 445 nM. As shown in FIGS. 16A and 16B, dose dependent I2S activity was observed in the liver, presented as fold liver I2S activity over wild-type (FIG. 16A; I2S activity in the liver of mice administered formulation buffer control), as well as in the serum, presented as fold serum I2S activity over wild-type (FIG. 16B). AAVHSC16-mediated I2S expression reached supraphysiologic levels of I2S activity in the liver at the 1E13 vg/kg dose and above, and supraphysiologic levels of I2S activity in the serum between the 1E13 vg/kg and 5E13 vg/kg doses. Taken together, this indicates that low levels of liver transduction can still lead to hepatic protein activity, secretion and cross-correction potential. Without being bound to any theory, transduction of other tissues may contribute to serum secretion.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. (canceled)

2. A recombinant adeno-associated virus (AAV) capsid protein, wherein:
the amino acid in the capsid protein corresponding to amino acid position 346 of SEQ ID NO: 14 is alanine; the amino acid in the capsid protein corresponding to amino acid position 501 of SEQ ID NO: 14 is isoleucine; the amino acid in the capsid protein corresponding to amino acid position 505 of SEQ ID NO: 14 is arginine; and/or the amino acid in the capsid protein corresponding to amino acid position 706 of SEQ ID NO: 14 is cysteine, wherein the capsid protein does not comprise the amino acid sequence of amino acids 203-736 of SEQ ID NOs: 11-15; or
the amino acid in the capsid protein corresponding to amino acid positions 346, 501, 505, and/or 706 of SEQ ID NO: 14 is not the wild type amino acid.

3. The recombinant AAV capsid protein of claim 2, wherein:
a) the amino acid in the capsid protein corresponding to amino acid position 501 of SEQ ID NO: 14 is isoleucine, and the amino acid in the capsid protein corresponding to amino acid position 505 of SEQ ID NO: 14 is arginine;
b) the amino acid in the capsid protein corresponding to amino acid position 501 of SEQ ID NO: 14 is isoleucine, and the amino acid in the capsid protein corresponding to amino acid position 706 of SEQ ID NO: 14 is cysteine;
c) the amino acid in the capsid protein corresponding to amino acid position 505 of SEQ ID NO: 14 is arginine, and the amino acid in the capsid protein corresponding to amino acid position 706 of SEQ ID NO: 14 is cysteine,
d) the amino acid in the capsid protein corresponding to amino acid position 501 of SEQ ID NO: 14 is isoleucine, the amino acid in the capsid protein corresponding to amino acid position 505 of SEQ ID NO: 14 is arginine, and the amino acid in the capsid protein corresponding to amino acid position 706 of SEQ ID NO: 14 is cysteine;
e) The recombinant AAV capsid protein of claim 1 or 2, wherein the amino acid in the capsid protein corresponding to amino acid position 346 of SEQ ID NO: 14 is alanine, the amino acid in the capsid protein corresponding to amino acid position 501 of SEQ ID NO: 14 is isoleucine, and the amino acid in the capsid protein corresponding to amino acid position 505 of SEQ ID NO: 14 is arginine;
f) the amino acid in the capsid protein corresponding to amino acid position 346 of SEQ ID NO: 14 is alanine, the amino acid in the capsid protein corresponding to amino acid position 501 of SEQ ID NO: 14 is isoleucine, and the amino acid in the capsid protein corresponding to amino acid position 706 of SEQ ID NO: 14 is cysteine;
g) the amino acid in the capsid protein corresponding to amino acid position 346 of SEQ ID NO: 14 is alanine, the amino acid in the capsid protein corresponding to amino acid position 505 of SEQ ID NO: 14 is arginine, and the amino acid in the capsid protein corresponding to amino acid position 706 of SEQ ID NO: 14 is cysteine; or
h) the amino acid in the capsid protein corresponding to amino acid position 346 of SEQ ID NO: 14 is alanine, the amino acid in the capsid protein corresponding to amino acid position 501 of SEQ ID NO: 14 is isoleucine, the amino acid in the capsid protein corresponding to amino acid position 505 of SEQ ID NO: 14 is arginine, and the amino acid in the capsid protein corresponding to amino acid position 706 of SEQ ID NO: 14 is cysteine.

4-10. (canceled)

11. The recombinant AAV capsid protein of claim 2, wherein the recombinant AAV capsid protein is a variant of a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein.

12. The recombinant AAV capsid protein of claim 2, comprising an amino acid sequence that is 95% identical to any one of the amino acid sequences of SEQ ID NOs: 1-30.

13. The recombinant AAV capsid protein of claim 2, comprising any one of the amino acid sequences of SEQ ID NOs: 31-411 and 453-1304.

14. An isolated polynucleotide encoding the recombinant AAV capsid protein of claim 2.

15. A vector comprising the polynucleotide of claim 14, wherein:
the vector is a plasmid;
the vector is an expression vector; and/or
the vector is a viral vector, optionally wherein the viral vector is a retrovirus vector, a herpes virus vector, a baculovirus vector, or an adenovirus vector.

16-18. (canceled)

19. A recombinant cell comprising the vector of claim 15.

20. A method of producing a recombinant AAV capsid protein, the method comprising culturing the recombinant cell of claim 19 under conditions where the polynucleotide is expressed, and the capsid protein is produced.

21. A recombinant adeno-associated virus (rAAV) comprising:
(a) a capsid comprising one or more of the recombinant AAV capsid protein of claim 2; and
(b) an rAAV genome.

22. The rAAV of claim 21, wherein:
the rAAV genome comprises a transgene, optionally wherein:
the transgene encodes a polypeptide;
the transgene encodes a miRNA, shRNA, siRNA, antisense RNA, gRNA, antagomir, miRNA sponge, RNA aptazyme, RNA aptamer, lncRNA, ribozyme, or mRNA; and/or
the transgene is operably linked to a transcriptional regulatory element; or
the rAAV genome comprises an editing genome.

23-26. (canceled)

27. A method for transducing a cell, the method comprising contacting the cell with the rAAV of claim 21 under conditions whereby the cell is transduced.

28. A method for delivering a transgene in a cell, the method comprising contacting the cell with the rAAV of claim 21 under conditions whereby the cell is transduced and the transgene is expressed.

29. A method for editing a target locus in a genome of a cell, the method comprising contacting the rAAV of claim 21 under conditions whereby the cell is transduced and the target locus is edited.

30-32. (canceled)

33. A packaging system for preparation of an rAAV, wherein the packaging system comprises:
(a) a first nucleotide sequence encoding one or more AAV Rep proteins;
(b) a second nucleotide sequence encoding the recombinant AAV capsid protein of claim 2; and
(c) a third nucleotide sequence comprising an rAAV genome sequence.

34-41. (canceled)

42. A method for recombinant preparation of an rAAV, the method comprising introducing the packaging system of claim 33 into a cell under conditions whereby the rAAV is produced.

43. (canceled)

44. A recombinant cell comprising the polynucleotide of claim 14.

45. A method of producing a recombinant AAV capsid protein, the method comprising culturing the recombinant cell of claim 44 under conditions where the polynucleotide is expressed, and the capsid protein is produced.
